(19) United States
(12) Patent Application Publication
(10) Pub. No.: US 2026/0325581 A1
(43) Pub. Date: Oct. 22, 2026

(54) PAP-Targeted CAR-T Cells and Antigen-Independent Expansion Systems for Solid Tumor Immunotherapy

(71) Applicants: Innovative Cellular Therapeutics Holdings, Ltd., George Town (KY); Innovative Cellular Therapeutics, Inc., Rockville, MD (US)

(72) Inventors: Zhiyuan Cao, Rockville, MD (US); Chengfei Pu, Shanghai (CN); Zhao Wu, Rockville, MD (US); Lei Xiao, Rockville, MD (US)

(73) Assignees: Innovative Cellular Therapeutics Holdings, Ltd., George Town (KY); Innovative Cellular Therapeutics, Inc., Rockville, MD (US)

(21) Appl. No.: 19/639,030

(22) Filed: Apr. 3, 2026

Related U.S. Application Data
(60) Provisional application No. 63/783,831, filed on Apr. 4, 2025.

(51) Int. Cl. A61K 35/17; A61K 9/00; A61K 38/19; A61K 40/11; A61K 40/13; A61K 40/31; A61K 40/42; A61P 35/04; C07K 14/705; C07K 16/40; G01N 33/68

(52) U.S. Cl. CPC A61K 35/17; A61K 9/0019; A61K 38/19; A61K 40/11; A61K 40/13; A61K 40/31; A61K 40/4211; A61K 40/4252; A61P 35/04; C07K 14/7051; C07K 14/70578; C07K 16/40; G01N 33/6863; G01N 33/6893; A61K 2239/13; A61K 2239/31; A61K 2239/58; C07K 2317/21; C07K 2317/622; C07K 2319/02; G01N 2333/52

(57) ABSTRACT

The present disclosure relates to a modular CAR-T cell therapy system for treating prostate cancer using a chimeric antigen receptor (CAR) targeting transmembrane prostatic acid phosphatase (PAP). To overcome the limitations of antigen-restricted expansion in solid tumors, the invention includes a CoupledCAR platform comprising CD19-targeting CAR-T cells and CD19⁺ B cells that deliver costimulatory and cytokine signals to PAP CAR-T cells in vivo. This antigen-independent expansion strategy enhances persistence, promotes a memory-like T cell phenotype, and reduces exhaustion and treatment-related toxicities. The system supports scalable, programmable CAR-T cell activation without tumor antigen exposure and may be used for broader applications in cellular immunotherapy.

### CROSS REFERENCE TO RELATED APPLICATIONS

This application claims the benefit of U.S. Provisional Application No. 63/783,831 filed Apr. 4, 2025, which is incorporated herein by reference in its entirety.

### TECHNICAL FIELD

The present disclosure relates to compositions and methods for expanding and maintaining modified cells including genetically modified cells and uses thereof in the treatment of diseases, including cancer.

### BACKGROUND

Chimeric antigen receptor (CAR)-T cell therapies have demonstrated remarkable success in hematologic malignancies, particularly those targeting CD19. However, translating CAR-T therapies to solid tumors remains a significant challenge due to limited tumor-specific antigens, poor T cell persistence, and an immunosuppressive tumor microenvironment. Existing CAR-T approaches often rely on sustained antigen engagement to drive expansion and therapeutic effect, which can also contribute to T cell exhaustion and on-target off-tumor toxicities.

Prostatic acid phosphatase (PAP) is a transmembrane protein with high specificity for prostate tissue and prostate tumors. Despite its potential as a target, previous PAP-directed immunotherapies have shown limited efficacy, and there remains a need for improved targeting strategies that enhance antitumor response while minimizing toxicity.

Additionally, conventional CAR-T therapies depend heavily on tumor antigen presence for in vivo expansion. In the solid tumor setting, this limits therapeutic durability and response, especially when antigen expression is heterogeneous or inaccessible.

### SUMMARY

Embodiments relate to a system for immune therapy, the system comprising: a first therapeutic cell population comprising T cells engineered to express a chimeric antigen receptor (CAR) that specifically binds to transmembrane prostatic acid phosphatase (PAP), a prostate cancer-associated surface antigen; a second therapeutic cell population comprising T cells engineered to express a CAR that binds an immune cell antigen such as CD19; and an auxiliary immune cell population comprising lymphocytes, such as B cells, that express the immune cell antigen. In embodiments, the second therapeutic cell population and the auxiliary immune cell population are configured to deliver costimulatory and cytokine signaling sufficient to induce in vivo expansion of the PAP-specific CAR-T cells in the absence of engagement with PAP antigen. In embodiments, the system enables antigen-independent CAR-T cell expansion for use in gene and/or cellular immunotherapy for prostate cancer and potentially other solid tumors. The system may be implemented as a modular, closed, and optionally automated therapeutic platform for enhancing CAR-T persistence, memory phenotype, and reduced toxicity in adoptive cell transfer therapy.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIGS. 1A-IC illustrate the identification of PAP as a novel prostate cancer-specific CAR-T target. Bulk RNA sequencing analysis (TCGA and GTEx) was used to evaluate tumor versus adjacent normal tissue expression, highlighting genes with high tumor-to-normal fold change and prostate specificity. Tissue-wide specificity across 54 tissue types confirmed restricted expression of candidate genes. Single-cell transcriptome data from seven prostate tumor patients were visualized using UMAP, with distinct cell type clustering and differential PAP expression in epithelial compartments. Violin plots revealed that PAP expression is enriched in tumor epithelial cells relative to other compartments and to normal epithelium. Tissue microarray-based immunohistochemistry (IHC) confirmed PAP protein expression in prostate tumors, with representative IHC images showing strong staining in tumor samples but not in control tissues. Comparative IHC scoring of PAP, PSMA, and CD46 further highlighted the specificity of PAP expression. These data collectively validate PAP as a highly prostate-specific CAR-T target.

FIGS. 2A-2I describe the selection and engineering of PAP-specific single-chain variable fragment (scFv) clones for CAR-T design. A recombinant transmembrane PAP (tmPAP) protein was used for initial antibody screening. Binding assays identified several human scFv clones, among which clone S5D1 exhibited high affinity to tmPAP, with kinetic binding analysis confirming low nanomolar KD. GFP-tagged CAR constructs were generated based on candidate scFvs, and surface expression was verified via flow cytometry. Functional assays showed that S5D1-based CAR-T cells mediated cytotoxicity against PAP-expressing targets and secreted IFNγ upon activation. In vivo efficacy was evaluated using a PC3-tmPAP xenograft model, with S5D1 CAR-T treated mice showing suppressed tumor growth and increased CAR-T cell persistence in spleen and tumor tissue.

FIGS. 3A-3D present the design and validation of the CoupledCAR system for antigen-independent expansion of PAP CAR-T cells. A schematic outlines the three-cell co-culture approach involving PAP CAR-T, CD19 CAR-T, and B cells. In vitro proliferation assays demonstrated that PAP CAR-T expansion is enhanced in the presence of CD19 CAR-T/B cell co-culture, even without PAP antigen. Flow cytometry confirmed that B cells upregulate costimulatory molecules (CD80 and CD86) following interaction with CD19 CAR-T cells. Cytokine profiling revealed increased levels of IL-2, IL-15, and CXCL9 in the CoupledCAR setting. Additional assays demonstrated that cytokine supplementation alone could recapitulate some, but not all, aspects of CoupledCAR-mediated proliferation. In vivo, the CoupledCAR approach led to significant expansion of PAP CAR-T cells, reduced tumor burden, and improved survival in xenograft-bearing mice, as evidenced by bioluminescence imaging and Kaplan-Meier survival analysis.

FIGS. 4A-4M explore the phenotypic and transcriptional impact of CoupledCAR expansion on PAP CAR-T cells. Single-cell RNA sequencing followed by UMAP dimensionality reduction revealed distinct T cell clusters with memory versus effector phenotypes. Gene signature analysis showed enrichment of central memory markers (e.g., TCF7, IL7R, CCR7) in CoupledCAR-expanded CAR-T cells. A heatmap of trafficking and differentiation genes supported this memory-biased phenotype. Conversely, exhaustion markers such as PDCD1, LAG3, and the NR4A family were suppressed. Pseudotime trajectory analysis confirmed progression toward memory phenotypes rather than exhaustion. Cell-cell communication analysis (CellChat) identified ligand-receptor interactions between CD19 CAR-T cells, B cells, and PAP CAR-T cells, implicating CD80/CD86 and cytokine-mediated pathways as drivers of expansion. A conceptual model summarizes how CoupledCAR delivers antigen-independent costimulatory and cytokine signals to maintain a durable, low-exhaustion CAR-T phenotype.

FIGS. 5A-5K demonstrates that the CoupledCAR approach enhances the expansion and functional performance of PAP CAR-T cells in vivo. A prostate cancer xenograft model was established by subcutaneous injection of PC3-tmPAP cells, followed by infusion of different T and B cell combinations 24 days later. Mice were grouped into nine cohorts, including those receiving PAP CAR-T cells alone or in combination with CD19 CAR-T cells and/or B cells. B cells were infused on days 3, 6, and 9 in applicable groups. Peripheral blood analysis on day 14 showed differential CAR-T cell expansion and cytokine secretion. Tumor volumes were tracked every three days, revealing superior suppression in the group receiving PAP CAR-T cells with both CD19 CAR-T and B cells. Immunohistochemistry revealed increased infiltration of CD3⁺, CD4⁺, CD8⁺, and PAP CAR-transduced T cells in tumors, with quantification shown in bar graphs. Statistical analyses were performed using t-tests and one-way ANOVA, with significance thresholds noted.

FIGS. 6A-6E focuses on the signaling mechanisms underlying PAP CAR-T expansion in the CoupledCAR system. The experimental design is depicted for single-cell RNA sequencing using 10× Genomics. Heatmaps illustrate the temporal dynamics of TCR signaling, costimulatory pathways, and cytokine/chemokine signaling across CD19 CAR-T cells, PAP CAR-T cells, and B cells at 0, 4, 24, and 48 hours. An additional heatmap highlights distinct phenotypic outcomes for CD19 and PAP CAR-T cells when activated through different mechanisms, demonstrating that the CoupledCAR system preferentially activates signaling pathways consistent with memory and persistence without exhaustion.

FIGS. 7A-7E provides a detailed analysis of cell-cell communication using the CellChat algorithm. Costimulatory and cytokine/chemokine interactions among CD19 CAR-T cells, PAP CAR-T cells, and B cells were profiled at 4, 24, and 48 hours, revealing dynamic ligand-receptor activity. Flow cytometry was used to assess surface expression of CD80 and CD86 in B cells after 24-hour co-culture with CD19 CAR-T cells at various effector-to-target ratios. The resulting data showed increases in CD80⁺, CD86⁺, and CD80⁺CD86⁺ B cell subsets, quantified in bar graphs. Statistical significance was evaluated via unpaired t-tests, with P values provided for each marker.

FIGS. 8A-8D demonstrates the expression specificity of prostatic acid phosphatase (PAP) in normal and tumor tissues. Analysis of the GTEx dataset reveals that PAP expression is highly restricted to prostate tissue in healthy individuals. TCGA data show elevated PAP expression in prostate tumor samples compared to other cancer types, with a clear differential between prostate adenocarcinoma (PRAD) tumors and matched normal tissues. Single-cell RNA sequencing of human prostate tumors confirms that PAP expression is localized primarily to the epithelial compartment. Immunohistochemistry further validates strong PAP protein expression in prostate tumor tissues, with minimal or no detectable expression in non-prostate control tissues. Comparative IHC scoring highlights the superior tumor specificity of PAP relative to other targets such as PSMA and CD46.

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies, monoclonal antibodies, and Fv, Fab, Fab′ and F(ab′)₂ and fragments, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full length antibody, for example, the antigen binding or variable region of the antibody. Other examples of antibody fragments include Fab, Fab′, F(ab′)₂, and Fv fragments; diabodies; linear antibodies; single-chain antibody molecules; and multi-specific antibodies formed from antibody fragments.

The term “Fv” refers to the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and X light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody, or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “autoantigen” or “self-antigen” refers to an antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be related or unrelated to the recipient subject, but the donor subject has immune system markers which are similar to the recipient subject.

The term “xenogeneic” is used to describe a graft derived from a subject of a different species. As an example, the donor subject is from a different species than a recipient subject and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Cancers that may be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast Cancer |
| CLCA1 | colorectal Cancer |
| MUC12 | colorectal Cancer |
| GUCY2C | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/ |
| | Urothelial cancer |
| UPK2 | Urothelial cancer |
| | (including bladder cancer) |
| ADAM12 | Breast cancer, |
| | pancreatic cancer and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL-13Rα2 | Glioma |
| (IL-13 receptor alpha 2) | |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomas |
| EpCAM | Carcinomas |
| EGFRvIII | Glioma-Glioblastoma |
| EGFR | Glioma-NSCL cancer |
| tMUC 1 | Cholangiocarcinoma, |
| | Pancreatic cancer, Breast |
| PSCA | pancreas, stomach, |
| | or prostate cancer |

Throughout this specification, unless the context requires otherwise, the words “comprise,” “includes” and “including” will be understood to imply the inclusion of a stated step or element (ingredient or component) or group of steps or elements (ingredients or components) but not the exclusion of any other step or element or group of steps or elements.

The phrase “consisting of” is meant to include, and is limited to, whatever follows the phrase “consisting of” Thus, the phrase “consisting of” indicates that the listed elements or steps are required or mandatory and that no other elements may be present.

The phrase “consisting essentially of” is meant to include any element listed after the phrase and can include other elements or steps that do not interfere with or contribute to the activity or action specified in the disclosure for the listed elements or steps. Thus, the phrase “consisting essentially of” indicates that the listed elements or steps are required or mandatory, but that other elements or steps are optional and may or may not be present depending upon whether or not they affect the activity or action of the listed elements or steps. In embodiments, those elements or steps that do not affect an embodiment are those elements or steps that do not alter the embodiment's ability in a statistically significant manner to perform a function in vitro or in vivo, such as killing cancer cells in vitro or in vivo.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The terms “co-stimulatory signaling region”, “co-stimulatory domain”, and “co-stimulation domain” are used interchangeably to refer to one or more additional stimulatory domain in addition to a stimulatory or signaling domain such as CD3 zeta. The terms “stimulatory” or “signaling” domain (or region) are also used interchangeably, when referring, for example, to CD3 zeta, the primary signaling domain. In embodiments, the co-stimulatory signaling domain and the stimulatory signaling domain can be on the same molecule or different molecules in the same cell.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for the synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter. The term “overexpression” refers to the production of a gene product in transgenic organisms or cells that exceeds levels of production in normal or non-transformed organisms or cells.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses (AAV)) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared x100. For example, if 6 of 10 of the positions in two sequences are matched or homologous then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially free from components that normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “parenteral administration” of a composition includes, e.g., subcutaneous (s.c.), intravenous (i.v.), intramuscular (i.m.), intrasternal injection, or infusion techniques.

The terms “patient,” “subject,” “individual,” and the like are used interchangeably herein, and refer to any animal, such as a mammal, for example, a human or any living organism amenable to the methods described herein. In embodiments, the patient, subject, or individual is a human or mammal. In embodiments, the term “subject” is intended to include living organisms in which an immune response can be elicited (e.g., mammals). Examples of subjects include humans, and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder. Accordingly, the subject can also be in need of prevention of a disease condition or disorder. In embodiments, the disease is cancer.

The term “polynucleotide” or “nucleic acid” refers to mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In embodiments, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In embodiments, a polypeptide variant is distinguished from a reference polypeptide by one or more substitutions, which may be conservative or non-conservative. In embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

“NFAT promoter” refers to one or more NFAT binding sites or motifs linked to a minimal promoter of any gene expressed by T cells. In embodiments, the minimal promoter of a gene expressed by T cells is a minimal human IL-12 promoter. NFAT (nuclear factor of activated T cells) are transcription factors. Examples of NFAT transcription factors include NFAT 1, NFAT2, NFAT3, NFAT4, and NFAT5. These transcription factors bind NFAT binding sites or motifs in the NFAT promoter. The NFAT promoter (or a functional portion or functional variant thereof) can comprise any number of binding motifs, e.g., at least two, at least three, at least four, at least five, or at least six, at least seven, at least eight, at least nine, at least ten, at least eleven, or up to twelve binding motifs. In embodiments, the NFAT promoter comprises six NFAT binding motifs. In an especially preferred embodiment, the NFAT promoter nucleotide sequence comprises or consists of a functional portion or functional variant thereof.

The NFAT promoter (or a functional portion or functional variant thereof) is operatively associated with the nucleotide sequence encoding IL-12 (or a functional portion or functional variant thereof). “Operatively associated with” means that the nucleotide sequence encoding IL-12 (or a functional portion or functional variant thereof) is transcribed into IL-12 mRNA when the NFAT protein binds to the NFAT promoter sequence (or a functional portion or functional variant thereof). Without being bound to a particular theory, it is believed that NFAT is regulated by a calcium signaling pathway. In particular, it is believed that TCR stimulation (by, e.g., an antigen) and/or stimulation of the calcium signaling pathway of the cell (by, e.g., PMA/Ionomycin) increases intracellular calcium concentration and activates calcium channels. It is believed that the NFAT protein is then dephosporylated by calmoduin and translocates to the nucleus where it binds the NFAT promoter sequence (or a functional portion or functional variant thereof) and activates downstream gene expression. By providing an NFAT promoter (or a functional portion or functional variant thereof) that is operatively associated with the nucleotide sequence encoding IL-12 (or a functional portion or functional variant thereof), the nucleic acids described herein advantageously make it possible to express IL-12 (or a functional portion or functional variant thereof) only when the host cell including the nucleic acid is stimulated by, e.g., PMA/Ionomycin and/or an antigen. More information can be found at U.S. Pat. No. 8,556,882, which is incorporated by the reference.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as being specific. In another example, an antibody that specifically binds an antigen may also bind different allelic forms of the antigen. However, such cross reactivity does not itself alter the classification of an antibody as being specific. In some instances, the terms “specific binding” or “specifically binding,” can be used to describe the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence, for example via engineering (altering one or more amino acids) of the recognition helix region of a naturally occurring zinc finger protein. Further, a Zinc finger binding domain may be fused a DNA-cleavage domain to form a Zinc finger nuclease (ZFN) targeting a specific desired DNA sequence. For example, a pair of ZFNs (e.g., a ZFN-left arm and a ZFN-right arm) may be engineered to target and cause modifications of specific desired DNA sequences (e.g., TRAC genes).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In embodiments, fusion polypeptides are used for targeted double-stranded DNA cleavage.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins (for example, a fusion between a ZFP DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage, and polypeptide ligation can also be involved in the expression of the protein in a cell. Methods for polynucleotide and polypeptide delivery to cells are presented elsewhere in this disclosure.

“Modulation” of gene expression refers to a change in the activity of a gene. Modulation of expression can include but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

By “statistically significant,” it is meant that the result was unlikely to have occurred by chance. Statistical significance can be determined by any method known in the art. Commonly used measures of statistical significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures. CD3 zeta is not the only suitable primary signaling domain for a CAR construct with respect to the primary response. For example, back in 1993, both CD3 zeta and FcRy were shown as functional primary signaling domains of CAR molecules. Eshhar et al., “Specific activation and targeting of cytotoxic lymphocytes through chimeric single chains consisting of antibody-binding domains and the gamma or zeta subunits of the immunoglobulin and T cell receptors” PNAS, 1993 Jan. 15; 90(2):720-4, showed that two CAR constructs in which an scFv was fused to “either the FcR gamma chain or the CD3 complex chain” triggered T cell activation and target cell. Notably, as demonstrated in Eshhar et al., CAR constructs containing only the primary signaling domain CD3 zeta or FcR gamma are functional without the co-presence of co-stimulatory domains. Additional non-CD3 zeta based CAR constructs have been developed over the years. For example, Wang et al. (“A Chimeric Antigen Receptor (CARs) Based Upon a Killer Immunoglobulin-Like Receptor (KIR) Triggers Robust Cytotoxic Activity in Solid Tumors” Molecular Therapy, vol. 22, no. Suppl.1, May 2014, page S57) tested a CAR molecule in which an scFv was fused to “the transmembrane and cytoplasmic domain of a killer immunoglobulin-like receptor (KIR). Wang et al. reported that, “a KIR-based CAR targeting mesothelin (SS 1-KIR) triggers antigen-specific cytotoxic activity and cytokine production that is comparable to CD3~-based CARs.” A second publication from the same group, Wang et al. (“Generation of Potent T-cell Immunotherapy for Cancer Using DAP12-Based, Multichain, Chimeric Immunoreceptors” Cancer Immunol Res. 2015 July; 3(7):815-26) showed that a CAR molecule in which “a single-chain variable fragment for antigen recognition was fused to the transmembrane and cytoplasmic domains of KIR2DS2, a stimulatory killer immunoglobulin-like receptor (KIR)” functioned both in vitro and in vivo “when introduced into human T cells with DAP12, an immunotyrosine-based activation motifs-containing adaptor.”

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor complex. The stimulatory molecule includes a domain responsible for signal transduction.

The term “stimulatory ligand” refers to a ligand that when present on an antigen presenting cell (e.g., an APC, a dendritic cell, a B-cell, and the like.) can specifically bind with a cognate binding partner (referred to herein as a “stimulatory molecule”) on a cell, for example a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to a treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with an exogenous nucleic acid. The cell includes the primary subject cell and its progeny.

The term “vector” refers to a polynucleotide that comprises an isolated nucleic acid that can be used to deliver the isolated nucleic acid to the interior of a cell. The cell can be an in vitro cell or an in vivo cell in a subject. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate transfer of nucleic acid into cells, such as, for example, polylysine compounds, liposomes, and the like. Examples of viral vectors include, adenoviral vectors, adeno-associated virus vectors, retroviral vectors, and others. For example, lentiviruses are complex retroviruses, which, in addition to the common retroviral genes gag, pol, and env, contain other genes with regulatory or structural functions. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

In embodiments, a polynucleotide encoding the antigen binding molecule and/or therapeutic agent(s) can be used to implement techniques described herein. The method or use includes: providing a viral particle (e.g., AAV, lentivirus or their variants) comprising a vector genome, the vector genome comprising the polynucleotide, wherein the polynucleotide is operably linked to an expression control element conferring transcription of the polynucleotide; and administering an amount of the viral particle to the subject such that the polynucleotide is expressed in the subject. In embodiments, the AAV preparation may include AAV vector particles, empty capsids and host cell impurities, thereby providing an AAV product substantially free of AAV empty capsids. More information of the administration and preparation of the viral particle may be found at the U.S. Pat. No. 9,840,719 and Milani et al., Sci. Transl. Med. 11, eaav7325 (2019) 22 May 2019, which are incorporated herein by reference. In embodiments, the polynucleotide may integrate into the genome of the modified cell and the progeny of the modified cell will also express the polynucleotide, resulting in a stably transfected modified cell. In embodiments, the modified cell expresses the polynucleotide encoding the CAR but the polynucleotide does not integrate into the genome of the modified cell such that the modified cell expresses the transiently transfected polynucleotide for a finite period of time (e.g., several days), after which the polynucleotide is lost through cell division or other factors. For example, the polynucleotide is present in the modified cell in a recombinant DNA construct, in an mRNA, or in a viral vector, and/or the polynucleotide is an mRNA, which is not integrated into the genome of the modified cell. In embodiments, the vector is a lentivirus. In embodiments, the lentivirus can be packaged with a particle (e.g., a Nano particle) such as to be released for a predetermined time and directly infused to the subject such that the lentivirus can be transferred to T cells of the subject.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In addition, unless otherwise indicated, numbers expressing quantities of ingredients, constituents, reaction conditions and so forth used in the specification and claims are to be understood as being modified by the term “about.”

The T cell response in a subject refers to cell-mediated immunity associated with a helper, killer, regulatory, and other types of T cells. For example, T cell response may include activities such as assistance to other white blood cells in immunologic processes and identifying and destroying virus-infected cells and tumor cells. T cell response in the subject may be measured via various indicators such as the number of virus-infected cells and/or tumor cells that T cells kill, an amount of cytokines that T cells release, for example, in co-culturing with virus-infected cells and/or tumor cells, a level of proliferation of T cells in the subject, a phenotype change of T cells (e.g., changes to memory T cells), and the longevity or lifespan of T cells in the subject.

In embodiments, in vitro killing assay may be performed by measuring the killing efficacy of CAR-T cells by co-culturing CAR-T cells with antigen-positive cells. CAR-T cells may be considered to have killing effect on the corresponding antigen-positive cells by showing a—decrease in the number of corresponding antigen-positive cells co-cultured with CAR-T cells and an increase in the release of cytokines such as IFN-7, TNF-α, and the like, as compared to control cells that do not express the corresponding antigen. Further, in vivo antitumor activity of the CAR-T cells may be tested. For example, xenograft models can be established using the antigens described herein in immunodeficient mice. Heterotransplantation of human cancer cells or tumor biopsies into immunodeficient rodents (xenograft models) has, for the past two decades, constituted the major preclinical screen for the development of novel cancer therapeutics (Song et al., Cancer Res. PMC 2014 Aug. 21, and Morton et al., Nature Protocols, 2, -247-250 (2007)). To evaluate the anti-tumor activity of CAR-T cells in vivo, immunodeficient mice bearing tumor xenografts were evaluated for CAR-T cell anti-tumor activity, for example, a decrease in mouse tumors and/or mouse blood cytokines, such as IFN-γ, TNF-α, and the like.

The term “chimeric antigen receptor” or alternatively a “CAR” refers to a recombinant polypeptide comprising at least an extracellular antigen binding domain, a transmembrane domain, and an intracellular domain (e.g., cytoplasmic domain) including an intracellular signaling domain. In embodiments, the domains in the CAR polypeptide are on the same polypeptide chain, for example, comprising a chimeric fusion protein. In embodiments, the domains of the CAR polypeptide are not on the same molecule, for example, not contiguous with each other, or are on different polypeptide chains.

In embodiments, the intracellular signaling domain may include a functional signaling domain derived from a stimulatory molecule and/or a co-stimulatory molecule as described herein. In embodiments, the intracellular signaling domain includes a functional signaling domain derived from a primary signaling domain (e.g., a primary signaling domain of CD3-zeta). In embodiments, the intracellular signaling domain further includes one or more functional signaling domains derived from at least one co-stimulatory molecule. The co-stimulatory signaling region refers to a portion of the CAR including the intracellular domain of a co-stimulatory molecule. Co-stimulatory molecules can include cell surface molecules for inducing an efficient response from the lymphocytes (in response to an antigen).

Between the extracellular domain and the transmembrane domain of the CAR, there can be incorporated a spacer domain. As used herein, the term “spacer domain” generally means any oligo- or polypeptide that functions to link the transmembrane domain to the extracellular domain and/or the cytoplasmic domain in the polypeptide chain. A spacer domain may include up to 300 amino acids, 10 to 100 amino acids, or 25 to 50 amino acids.

The extracellular domain of a CAR may include an antigen binding domain (e.g., a scFv, a single domain antibody, or TCR, such as a TCR alpha binding domain or a TCR beta binding domain), that targets a specific tumor marker (e.g., a tumor antigen). Tumor antigens are proteins that are produced by tumor cells that elicit an immune response, particularly T cell mediated immune responses. Tumor antigens are well known in the art and include, for example, a glioma-associated antigen, carcinoembryonic antigen (CEA), 3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. For example, when the antigen that the CAR binds is CD19, the CAR thereof is referred to as CD19 CAR. 19CAR, CD19CAR, CD19 CAR, or CD19-CAR), which is a CAR molecule that includes an antigen binding domain that binds CD19.

In embodiments, the extracellular ligand-binding domain comprises a scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)₃ (SEQ ID: 2), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides comprising about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor a 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-a, CD44v7/8, 8H9, NCAM, VEGF receptors, 5T4, Fetal AchR, NKG2D ligands, CD44v6, TEM1, TEM8, or viral-associated antigens expressed by a tumor. In embodiments, the binding element of the CAR includes any antigen binding moiety that when bound to its cognate antigen, affects a tumor cell such that the tumor cell fails to grow, decrease in size, or dies.

The CAR can be a bispecific CAR. For example, the two antigen binding domains are on the same CAR (a bispecific CAR or tandem CAR (tanCAR)), on different CAR molecules, or on a CAR and T cell receptor (TCR). A single CAR can include two different antigen binding domains, or the two different antigen binding domains are each on a separate CAR. The CAR can have more than two antigen binding domains, for example, a multispecific CAR. The antigen binding domains of the multispecific CAR can be on the same CAR or on separate CAR, such as one antigen binding domain on each CAR.

In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the intracellular domain comprises a CD3 zeta signaling domain. Embodiments relate to a vector comprising the isolated nucleic acid sequence described herein. Embodiments relate to an isolated cell comprising the isolated nucleic acid sequence described herein.

The cells, including CAR cells and modified cells, described herein can be derived from a stem cell. The stem cells may be adult stem cells, embryonic stem cells, or non-human stem cells, cord blood stem cells, progenitor cells, bone marrow stem cells, induced pluripotent stem cells, totipotent stem cells, or hematopoietic stem cells. The cells can also be a dendritic cell, a NK-cell, a B-cell, or a T cell selected from the group consisting of inflammatory T lymphocytes, cytotoxic T lymphocytes, regulatory T lymphocytes, and helper T lymphocytes. In embodiments, the cells can be derived from the group consisting of CD4+ T-lymphocytes and CD8+ T-lymphocytes. Prior to expansion and genetic modification of the cells described herein, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T cell lines available and known to those skilled in the art, can be used. In embodiments, the cells may be derived from a healthy donor, from a patient diagnosed with cancer, or from a patient diagnosed with an infection. In embodiments, the cells are part of a mixed population of cells which present different phenotypic characteristics.

A population of cells refers to a group of two or more cells. The cells of the population could be the same, such that the population is a homogenous population of cells. The cells of the population could be different, such that the population is a mixed population or a heterogeneous population of cells. For example, a mixed population of cells could include modified cells comprising a first CAR and cells comprising a second CAR, wherein the first CAR and the second CAR bind different antigens.

The term “stem cell” refers to any type of cell which has the capacity for self-renewal and the ability to differentiate into other kind(s) of cell. For example, a stem cell gives rise either to two daughter stem cells (as occurs in vitro with embryonic stem cells in culture) or to one stem cell and a cell that undergoes differentiation (as occurs e.g. in hematopoietic stem cells, which give rise to blood cells). Different categories of stem cells may be distinguished on the basis of their origin and/or on the extent of their capacity for differentiation into other types of cell. Stem cells can include embryonic stem (ES) cells (i.e., pluripotent stem cells), somatic stem cells, induced pluripotent stem cells, and any other types of stem cells.

Pluripotent embryonic stem cells can be found in the inner cell mass of a blastocyst and have high innate capacity for differentiation. For example, pluripotent embryonic stem cells have the potential to form any type of cell in the body. When grown in vitro for long periods of time, ES cells maintain pluripotency, and progeny cells retain the potential for multilineage differentiation.

Somatic stem cells can include fetal stem cells (from the fetus) and adult stem cells (found in various tissues, such as bone marrow). These cells have been regarded as having a capacity for differentiation lower than that of the pluripotent ES cells—with the capacity of fetal stem cells being greater than that of adult stem cells; they apparently differentiate into only a limited number of different types of cells and have been described as multipotent. “Tissue-specific” stem cells normally give rise to only one type of cell. For example, embryonic stem cells can differentiate into blood stem cells (e.g., Hematopoietic stem cells (HSCs)), which can further differentiate into various blood cells (e.g., red blood cells, platelets, white blood cells, etc.).

Induced pluripotent stem cells (iPS cells or iPSCs) can include a type of pluripotent stem cell artificially derived from a non-pluripotent cell (e.g., an adult somatic cell) by inducing expression of specific genes. Induced pluripotent stem cells are similar to naturally occurring pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be isolated from adult stomach, liver, skin, and blood cells.

In embodiments, the CAR cells, the modified cell, or the cell is a T cell, a NK cell, a macrophage or a dendritic cell. For example, the CAR cells, the modified cell, or the cell is a T cell.

T cells, or T lymphocytes, are a type of white blood cell of the immune system. There are various types of T cells including T helper (TH) cells, cytotoxic T (TC) cells (T killer cells, killer T cells), natural killer T (NKT) cells, memory T (Tm) cells, regulatory T (Treg) cells, and gamma delta T (γδ T) cells.

T helper (TH) cells assist other lymphocytes, for example, activating cytotoxic T cells and macrophages and maturation of B cells into plasma cells and memory B cells. These T helper cells express CD4 glycoprotein on their surface and are also known as CD4+ T cells. Once activated, these T cells divide rapidly and secrete cytokines.

Cytotoxic T (TC) cells destroy virus-infected cells and tumor cells and are also involved in transplant rejection. They express CD8 protein on their surface. Cytotoxic T cell release cytokines.

Natural Killer T (NKT) cells are different from natural killer cells. NKT cells recognize glycolipid antigens presented by CD1d. Once activated, NKT cells produce cytokine and release cell killing molecules.

Memory T (Tm) cells are long-lived and can expand to large number of effector T cells upon re-exposure to their cognate antigen. Tm cells provide the immune system with memory against previously encountered pathogens. There are various subtypes of Tm cells including central memory T (TCM) cells, effector memory T (TEM) cells, tissue resident memory T (TRM) cells, and virtual memory T cells. Tm cells are either CD4+ or CD8+ and usually CD45RO.

Regulatory T (Treg) cells shut down T cell mediated immunity at the end of an immune reaction and suppress autoreactive T cells that escaped the process of negative selection in the thymus. Subsets of Treg cells include thymic Treg and peripherally derived Treg. Both subsets of Treg require the expression of the transcription factor FOXP3.

Gamma delta T (γδ T) cells are a subset of T cells that possess a γδ T cell receptor (TCR) on the cell surface, as most T cells express the αβ TCR chains. γδ T cells are less common in human and mice and are mainly found in the gut mucosa, skin, lung, and uterus. They are involved in the initiation and propagation of immune responses.

In embodiments, the antigen binding molecule is a T Cell Receptor (TCR). In embodiments, the TCR is modified TCR. In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains.

In embodiments, a T cell clone that expresses a TCR with high affinity for the target antigen may be isolated. In embodiments, tumor-infiltrating lymphocytes (TILs) or peripheral blood mononuclear cells (PBMCs) may be cultured in the presence of antigen-presenting cells (APCs) pulsed with a peptide representing an epitope known to elicit a dominant T cell response when presented in the context of a defined HLA allele. High-affinity clones may be then selected on the basis of MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle (e.g., a gammaretrovirus or lentivirus) may be then generated and tested for functionality (antigen specificity and functional avidity) and used to produce a clinical lot of the vector. An aliquot of the final product is then used to transduce the target T cell population (generally purified from patient PBMCs), which is expanded before infusion into the subject.

In embodiments, the APCs include dendritic cells, macrophages, Langerhans cells and B cells, or T cells.

In embodiments, the binding element of the CAR may include any antigen binding moiety that when bound to its cognate antigen, affects a tumor cell for example, it kills the tumor cell, inhibits the growth of the tumor cell, or promotes death of the tumor cell.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned.

The embodiments of the present disclosure further relate to vectors in which a nucleic acid described herein is inserted. Vectors can be derived from retroviruses such as the lentivirus that are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Viruses can be used to deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for deliverying nucleic acids into a cell, for example, electroporation, gene gun, sonoporation, magnetofection, and the use of oligonucleotides, lipoplexes, dendrimers, and inorganic nanoparticles.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence.

Additional information related to expression of synthetic nucleic acids encoding CARs and gene transfer into mammalian cells is provided in U.S. Pat. No. 8,906,682, incorporated by reference in its entirety.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, “therapeutic amount”, or “effective amount” is indicated, the precise amount of the compositions of the present disclosure to be administered can be determined by a physician with consideration of individual differences in age, weight, tumor size, extent of infection or metastasis, and condition of the patient (subject). It can be stated that a pharmaceutical composition comprising the T cells described herein may be administered at a dosage of 10⁴ to 10⁹cells/kg body weight, preferably 10⁵ to 10⁶ cells/kg body weight, including all integer values within those ranges. T cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art by monitoring the patient for signs of disease and adjusting the treatment accordingly. In embodiments, activated T cells are administered to a subject and then subsequently blood is redrawn (or have apheresis performed). T cells are collected, expanded, and reinfused into the subject. This process can be carried out multiple times every few weeks. In embodiments, T cells can be activated from blood draws of from 10 cc to 400 cc. In embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, certain populations of T cells can be selected.

The administration of the pharmaceutical compositions described herein can be carried out in any convenient manner, including by aerosol inhalation, injection, ingestion, transfusion, implantation or transplantation. The pharmaceutical compositions described herein can be administered to a patient subcutaneously, intradermally, intratumorally, intranodally, intramedullary, intramuscularly, intravenously (i. v.), or intraperitoneally. In embodiments, the T cell compositions of the present disclosure are administered to a patient by intradermal or subcutaneous injection. In embodiments, the T cell compositions of the present disclosure are administered by i.v. injection. The compositions of T cells may be injected directly into a tumor, lymph node, or site of infection. In embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present disclosure may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In embodiments, the cell compositions of the present disclosure are administered following B-cell ablative therapy such as agents that react with CD20, e.g., Rituxan®. For example, subjects may undergo standard treatment with high dose chemotherapy followed by peripheral blood stem cell transplantation. In embodiments, following the transplant, subjects receive an infusion of the expanded immune cells of the present disclosure. In embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician depending on various factors.

Additional information on the methods of cancer treatment using engineered or modified T cells is provided in U.S. Pat. No. 8,906,682, incorporated by reference in its entirety.

In embodiments, the population of cells described herein is used in autologous CAR-T cell therapy. In embodiments, the CAR-T cell therapy is allogenic CAR-T cell therapy, TCR-T cell therapy, and NK cell therapy.

Embodiments relate to an in vitro method for preparing modified cells. The method may include obtaining a sample of cells from the subject. For example, the sample may include T cells or T cell progenitors. The method may further include transfecting the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

As used herein, the term “gene fusion” refers to the fusion of at least a portion of a gene to at least a portion of an additional gene. The gene fusion need not include entire genes or exons of genes. In some instances, gene fusion is associated with alternations in cancer. A gene fusion product refers to a chimeric genomic DNA, a chimeric messenger RNA, a truncated protein or a chimeric protein resulting from a gene fusion. The gene fusion product may be detected by various methods described in U.S. Pat. No. 9,938,582, which is incorporated as a reference herein. A “gene fusion antigen” refers to a truncated protein or a chimeric protein that results from a gene fusion. In embodiments, an epitope of a gene fusion antigen may include a part of the gene fusion antigen or an immunogenic part of another antigen caused by the gene fusion. In embodiments, the gene fusion antigen interacts with, or is part of, cell membranes.

In embodiments, detection of mRNA and protein expression levels of a target molecules (e.g., CARs and cytokines) in cells, such as human cells, may be performed using experimental methods such as qPCR and FACS. Further, target molecules specifically expressed in the corresponding tumor cells with very low expression or undetectable expression in normal tissue cells may be identified.

In embodiments, In Vitro Killer Assay as well as killing experiment of CAR-T Cells Co-Cultured with Antigen-Positive Cells can be performed. CAR-T cells can exhibit a killing effect on the corresponding antigen-positive cells, a decrease in the number of corresponding antigen-positive cells co-cultured with CAR-T cells, and an increase in the release of IFN-γ, TNF-α, etc. as compared to control cells that did not express the corresponding antigen.

In embodiments, In Vivo Killer Assay can be performed. For example, mice may be transplanted with corresponding antigen tumor cells, and tumorigenic, transfusion of CAR-T cells, and a decrease in mouse tumors and mouse blood IFN-γ, TNF-α, and other signals can be defected.

Embodiments relate to a method of eliciting and/or enhancing T cell response in a subject having a solid tumor or treating a solid tumor in the subject, the method comprising administering an effective amount of T cells comprising the CAR described herein. In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the intracellular domain comprises a CD3 zeta signaling domain.

Embodiments relate to a vector comprising the isolated nucleic acid described herein.

Embodiments relate to an isolated cell comprising the isolated nucleic acid sequence described herein. Embodiments relate to a composition comprising a population of T cells comprising the CAR described herein. Embodiments relate to a CAR encoded by the isolated nucleic acid sequence described herein. Embodiments relate to a method of eliciting and/or enhancing T cell response in a subject or treating a tumor of the subject, the method comprising: administering an effective amount of T cell comprising the CAR described herein.

In embodiments, the CAR molecules described herein comprise one or more complementarity-determining regions (CDRs) for binding an antigen of interest. CDRs are part of the variable domains in immunoglobulins and T cell receptors for binding a specific antigen. There are three CDRs for each variable domain. Since there is a variable heavy domain and a variable light domain, there are six CDRs for binding an antigen. Further since an antibody has two heavy chains and two light chains, an antibody can have twelve CDRs altogether for binding antigens.

In embodiments, the modified cells described herein includes a CAR molecule comprising at least two different antigen binding domains. The CAR molecule can be a bispecific CAR molecule. For example, the two antigen binding domains can be on the same CAR molecule, on different CAR molecules, or on a CAR molecule and T cell receptor (TCR). A single CAR can include at least two different antigen binding domains, or the two different antigen binding domains are each on a separate CAR molecule. The at least two different antigen binding domains can be on the same CAR molecule or different CAR molecules, but in the same modified cell. Moreover, the at least two different antigen binding domains can be on a CAR molecule and a T cell receptor in the same modified cell. In embodiments, the bispecific CAR molecule can include a binding domain binding an antigen of WBC (e.g., CD19) and a binding domain binding a solid tumor antigen. In embodiments, the bispecific CAR molecule may include two binding domains binding two different solid tumor antigens.

In embodiments, the at least two different antigen binding domains are on different CAR molecules which are expressed by different modified cells. Further, the one or more different antigen binding domains are on a CAR molecule and a T cell receptor, which are expressed by different modified cells.

Embodiments relate to a system for immune therapy, the system comprising: a sample or blood processing module configured to obtain CD3+ cells from a blood sample of a subject, for example, a blood collection apparatus; a cell incubation module configured to activate CD3+ cell and introduce a vector into the CD3+ cells, for example, a blood processing apparatus, and a cell infusion module configured to infuse at least a portion of the CD3+ cells into the subject, for example, a cell processing apparatus.

The sample or blood processing module can further comprise a unit configured to obtain cells from the peripheral blood of a subject, for example a human patient. For example, the unit is an apheresis device in which the blood of a person is passed through an apparatus that separates out one particular blood component and returns the remainder of the blood into the systemic circulation of the subject.

In embodiments, the sample separation module can purify CD3+ cells by negative selection using, for example, the RosetteSep T cell enrichment Cocktail or positive selection using anti-CD3 coupled to magnetic beads. Following isolation, CD3+ cells were cultured in X-VIVO 15 (Cambrex, Walkersville, MD) supplemented with 5% normal human AB serum (Valley Biomedical, Winchester, VA), 2 mM L-glutamine (Cambrex), 20 mM HEPES (Cambrex), and IL2 (100 units/mL; R&D Systems). In embodiments, the blood sample can be diluted using Dulbecco phosphate buffered saline (DPBS), and apheresis density gradient centrifugation can be performed using the sample separation module to obtain PBMCs containing lymphocytes. MACS buffer can be used to rinse the PBMCs, and Pan T Cell-Ab cocktail can be mixed with PBMCs and incubated for 5 min. Pan T cell beads cocktail can be added and incubated at 2-8 degrees for 10 min. In embodiments, the sample separation module can include or be coupled to LS columns used to collect CD3+ cells. In embodiments, the output port can be configured to infuse the remaining components of the blood sample or non-desired cells (e.g., CD3− cells) back to the subject and to flow the desired cells (CD3+ cells) to the cell incubation module.

In embodiments, the systems described herein comprise one or more modules, apparatuses and/or devices. In embodiments, the modules can comprise one or more apparatuses and/or devices.

As used herein, the term “flow” or “flowing” with respect to the cells, components of the blood, and/or fluids, refers to moving or transporting them from one location to another.

The cell incubation module can be configured for stimulation/activation and culturing of the CD3+ cells. For example, the CD3+ cells can be stimulated/activated with magnetic beads precoated with agonist antibodies against CD3 and CD28 at a ratio of three beads per cell, and then resuspended at a concentration of 106 CD+3 cells/mL for expansion for a predetermined time (e.g., 2-20 hours). The stimulated/activated CD3+ cells can be then transduced with vectors including nucleic acids encoding CD19-BBζ CAR (CD19 4-1BB CD3 zeta CAR) hours after the cell stimulation/activation and expansion for a predetermined time, for example, 1-5 hours, or harvested at specific time points for analysis. CD3+ cells can be maintained in culture at a concentration of 0.1 to 1×10⁶ cells/mL by adjusting the concentration based on cell counting by flow cytometry using antibodies to human CD4 and CD8. After completion of cell culture, the magnetic beads can be removed and tested for release criteria specified for T cell phenotype, cell viability (≥70%), concentration (≥80% CD3+CD45+), and transduction efficiency (≥2%). In embodiments, CD3+ cells can be co-cultured with beads conjugated with CD19 antigen comprising a His-tag, for example, 6-histidines attached to the C-terminus of the CD19 antigen, after removal of beads conjugated with anti-CD3 and anti-CD28 and transduction with lentiviral virus containing a nucleic acid encoding an anti-CD19 CAR, for example, CD19 CAR.

The cell infusion module can comprise an input port, an output port, and a rotating container. The input port can be configured to obtain the CD3+ cells from the cell incubation module; the rotating container can be configured to remove beads, for example, magnetic beads precoated with agonist antibodies against CD3 and CD28 or CD19 beads, and to replace culture media of the CD+3 cells with media suitable for infusion; and the output port can be configured to provide the CD+3 cell for the infusion to the subject.

In embodiments, the sample processing module is coupled or connected to the cell incubation module that is coupled or connected to the cell infusion module such that the CD3+ cells flow through the system starting from the subject and ending back to the subject.

In embodiments, the sample processing module comprises: an input port configured to receive the blood sample from the subject; a sample separation module configured to: separate the CD3+ cells from remaining components of the blood sample, and collect the CD3+ cells; and an output port configured to flow the CD3+ cells to the cell incubation module, and flow the remaining components of the blood sample back into the subject.

In embodiments, the sample separation module is configured to contact the blood sample with a composition comprising a CD3 aggregation reagent comprising CD3 recognizing moiety coupled to a magnetic bead; apply gravity sedimentation for sedimentation of CD3+ cells, and a magnetic field gradient to the blood sample for immobilizing the magnetic bead simultaneously; generate a pellet and a supernatant phase; and recover the desired cells from the pellet. In embodiments, the sample separation module is configured to contact the blood sample with a composition comprising molecules that recognize and specifically bind undesired blood components; simultaneously apply gravity sedimentation for sedimentation of undesired blood components, and a magnetic field gradient to the blood sample for immobilizing the magnetic bead; generate a pellet and a supernatant phase; and recover the desired cells from the supernatant phase.

In embodiments, the blood components can include, for example, plasma, red blood cells, white blood cells, platelets, immune cells, proteins such as antibodies and clotting factors, and vitamins.

In embodiments, the system comprises components that are composed of plastic. Examples of such components include storage bags, tubes and such as inlet and outlet tubes. The plastic can comprise polystyrene, polyvinylchloride, polycarbonate, glass, polyacrylate, polyacrylamide, polymethylmethacrylate (PMMA), polyethylene terephthalate (PET), polytetrafluoroethylene (PTFE), thermoplastic polyurethane (TPU), silicone, polyethylene (PE), polylactide (PLA), polyglycolide (PGA), and copolymers thereof. In embodiments, the system comprises components comprising collagen, chitin, alginate, hyaluronic acid, and hyaluronic acid derivatives, In embodiments, the components of the system comprise ceramics, glass materials, hydroxyapatite (HA), and calcium phosphate. In embodiments, the system comprises compositions comprising one or more of the above-mentioned materials.

In embodiments, the system further comprises one or more sensors configured to monitor the separation of the blood sample obtained from the subject, in particular by detecting the formation of layers of the blood sample, the change of pH value of the blood sample, and/or the change in temperature of the blood sample.

In embodiments, the cell incubation module comprises a rotating container configured to culture cells and/or grow cells. The rotating container is disposable and/or has been sterilized.

Embodiments relate to a method of immune therapy by implementing the system, the method comprising: obtaining peripheral blood from a subject; collecting CD3+ cells from the peripheral blood; returning the remaining components of the peripheral blood to the subject; obtaining vectors comprising a nucleic acid encoding a CAR and one or more agents that activate cells, for example, T cells; mixing the vectors with the obtained CD3+ cells and incubating the mixture for a predetermined time to introduce the nucleic acid into the CD3+ cells; replacing culture media of the transfected CD3+ cells with a solution suitable for infusion into the subject; and infusing at least a portion of the solution containing the transfected CD3+ cells into the subject.

In embodiments, the entire method or process is completed in less than 1, 2, 3, 4, or 5 days.

Embodiments relate to a method for sample processing using the sample processing apparatus, the method comprising: obtaining a blood sample from a subject; isolating desired cells from the blood sample; flowing the desired cells for further processing; returning the remaining components of the blood sample to the subject; introducing a nucleic acid encoding an antigen binding molecule into the desired cells to obtain transfected cells; and administering an effective amount of a composition comprising the transfected cells to a subject in need thereof. In embodiments, the desired cells are CD3+ cells.

Embodiments relate to a method of delivering a therapeutic protein to the peripheral blood system of a subject, the method comprising: administering to the peripheral blood system of the subject, a viral vector, for example, retrovirus, adenovirus, adeno associated virus, and/or Lentivirus vectors, comprising a nucleic acid encoding the therapeutic protein, such that cells, organs, or tissues of the peripheral vascular system of the subject express the therapeutic protein. The therapeutic protein can be an antigen binding molecule.

Embodiments relate to a method of treating cancer in a subject, the method comprising: administering to the peripheral blood system of the subject a viral vector comprising a nucleic acid encoding a CAR molecule such that cells, organs or tissues of the peripheral vascular system of the subject express the CAR molecule; and monitoring cell response, for example, T cell response, resulting from the expression of CAR molecule in the subject.

Embodiments relate to a method of in vivo expression of antigen binding molecule in lymphocytes of a subject, the method comprising: administering to the peripheral blood system of the subject a viral vector comprising a nucleic acid encoding an antigen binding molecule such that cells, organs, or tissues of the peripheral vascular system of the subject express the antigen binding molecule.

Examples of a viral vector includes an adeno associated virus (AAV) vector. AAV is a small nonpathogenic virus of the parvoviridae family. AAV is distinct from other members of this family by its dependence upon a helper virus for replication. In the absence of a helper virus, AAV can integrate in a locus-specific manner into the q arm of chromosome 19. Approximately, 5 kb genome of AAV consists of one segment of single-stranded DNA of either plus or minus polarity. The ends of the genome are short inverted terminal repeats which can fold into hairpin structures and serve as the origin of viral DNA replication. Physically, the parvovirus virion is non-enveloped, and its icosahedral capsid is approximately 20-30 nm in diameter.

The adeno-associated virus (AAV) capsid includes three related proteins, VP1, VP2, and VP3. VP3, the shortest of the three proteins, has a molecular weight of about 59 to 61 kDa and a sequence of about 524 to 544 amino acids. The sequence of VP3 is shared by all three VPs and is known as the VP3 common region. VP2, which is about 57 amino acids longer than VP3, has a molecular weight of about 64 to 67 kDa and a sequence of about 580 to 601 amino acids. The N-terminal region of the VP2 sequence is shared by VP1 and is known as the VP1/VP2 common region. VP1, which is about 137 amino acids longer than VP2, has a molecular weight of about 79 to 82 kDa and a sequence of about 713 to 738 amino acids. The N-terminal region of the VP1 sequence is unique to VP1 and is known as the VP1 unique (VP1u) region. These three capsid proteins, VP1, VP2, and VP3, are typically present in the capsid at a ratio approximating 1:1:10, respectively, although this ratio, particularly the amount of VP3, can vary significantly and should not be considered as limiting in any respect.

The ends of the AAV genome have short inverted terminal repeats (ITR), which have the potential to fold into T-shaped hairpin structures that serve as the origin of viral DNA replication. Within the ITR region, two elements have been described, which are central to the function of the ITR, a GAGC repeat motif and the terminal resolution site (TRS). The GAGC repeat motif has been shown to bind Rep (replication protein) when the ITR is in either a linear or hairpin conformation. This binding serves to position Rep68/78 for cleavage at the TRS, which occurs in a site- and strand-specific manner. In addition to their role in replication, the GAGC repeat motif and the TRS appear to be central to viral integration. Contained within the chromosome 19 integration locus is a Rep binding site with an adjacent TRS. The GAGC repeat motif and the TRS have been shown to be functional for locus-specific integration. AAV are useful as gene therapy vectors as they can penetrate cells and introduce nucleic acid/genetic material so that the nucleic acid/genetic material can be stably maintained in cells.

In addition, these adeno-associated viruses (AAVs) can introduce nucleic acid/genetic material into specific sites, for example, a specific site on chromosome 19. Because AAV is not associated with pathogenic disease in humans, AAV vectors are able to deliver heterologous polynucleotide sequences encoding therapeutic proteins and agents to human patients without causing substantial AAV pathogenesis or disease. Accordingly, recombinant AAV (rAAV) vectors, including serotypes and variants, provide a means for the delivery of nucleic acids encoding proteins into cells ex vivo, in vitro, and in vivo, such that the cells express the encoded proteins. For example, an rAAV vector can include a heterologous nucleic acid encoding a desired protein or peptide. Vector delivery or administration to a subject, such as a mammal, provides the encoded protein to the subject. More Information on gene therapy and its clinical uses can be found at U.S. Pat. Nos. 9,433,688, 9,644,216, and 10,113,183; as well as Milone, M. C., O'Doherty, U. Clinical use of lentiviral vectors, Leukemia 32, 1529-1541 (2018); Rodrigues, G. A., Shalaev, E., Karami, T. K. et al. Pharmaceutical Development of AAV-Based Gene Therapy Products for the Eye. Pharm Res 36, 29 (2019); and Marquez Loza, L. I.; Yuen, E. C.; McCray, P. B., Jr. Lentiviral Vectors for the Treatment and Prevention of Cystic Fibrosis Lung Disease. Genes 2019, 10, 218; which are incorporated by reference in their entirety.

Modified AAV vectors, such as recombinant nucleic acids and polypeptides, are AAV vectors whose sequences have been manipulated, for example engineered or recombined, in a manner that the vector or sequence does not occur in nature. A particular example of a recombinant AAV vector would be one in which a nucleic acid that is not normally present in the wild-type viral genome, such as the AAV, genome, and is inserted within the viral genome and is heterologous to the viral genome. A “recombinant” AAV vector is distinguished from an AAV genome since all, or a part of the viral genome has been replaced with a non-native sequence with respect to the AAV genomic nucleic acid, such as a heterologous nucleic acid sequence. Although the term “recombinant” is not always used herein in reference to AAV vectors, as well as its sequences such as nucleic acids and polypeptides, recombinant forms of AAV, and sequences including nucleic acids and polypeptides, are expressly included in spite of any such omission. Typically for AAV, one or both inverted terminal repeat (ITR) sequences of the AAV genome are retained in the AAV vector. Incorporation of a non-native sequence therefore defines the AAV vector as a “recombinant” AAV (rAAV) vector.

A recombinant AAV vector can be packaged as a “particle” for subsequent transduction of a cell, for example, ex vivo, in vitro, or in vivo transduction. Where a recombinant AAV vector is encapsidated or packaged into an AAV particle, the particle can also be referred to herein as an “rAAV.” Such particles include proteins that encapsidate or package the vector genomes, and in the case of AAV, capsid proteins. An “AAV viral particle” or “AAV particle” refers to a viral particle composed of at least one AAV capsid protein, though typically all three capsid proteins of an AAV, and an encapsidated nucleic acid, referred to as a vector genome. If the particle comprises heterologous nucleic acid, it is typically referred to as “rAAV.”

An AAV vector “genome” refers to the portion of the recombinant plasmid sequence that is ultimately packaged or encapsidated to form an AAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant AAV production but is not itself packaged or encapsidated into rAAV particles.

In embodiments, rAAV vectors include capsids derived from AAV1, AAV2, AAV3, AAV4, AAV5, AAV6, AAV7, AAV8, AAV9, AAV10, AAV11, RhlO, Rh74, or AAV-2i8, as well as variants thereof, for example, capsid variants having amino acid insertions, additions and substitutions. AAV vector serotypes and variants include capsid variants, such as LK03, 4-l, and the like. rAAV serotypes and rAAV variants may or may not be distinct from other AAV serotypes (e.g., distinct from VP1, VP2, and/or VP3 sequences). The term “serotype” is a distinction used to refer to an AAV having a capsid that is serologically distinct from other AAV serotypes. Serologic distinctiveness is determined based on the lack of cross-reactivity between antibodies to one AAV as compared to another AAV. Such cross-reactivity differences are usually due to differences in capsid protein sequences/antigenic determinants for example, due to VP1, VP2, and/or VP3 sequence differences of AAV serotypes. Despite the possibility that AAV variants, including capsid variants, may not be serologically distinct from a reference AAV or other AAV serotype, the AAV variants differ by at least one nucleotide or amino acid residue from the reference or other AAV serotype.

Recombinant AAV vector, as well as methods and uses thereof, include any viral strain or serotype. As a non-limiting example, a recombinant AAV vector genome can be based upon any AAV genome, such as AAV-1, -2, -3, -4, -5, -6, -7, -8, 9, -10, -11, -rh74, -rhlO, or AAV-2i8. Such vectors can be based on the same strain or serotype (or subgroup or variant) or be different from each other. As a non-limiting example, a recombinant AAV vector genome based upon one serotype genome can be identical to one or more of the capsid proteins that package the vector. In addition, a recombinant AAV vector genome can be based upon an AAV, for example, AAV2, serotype genome distinct from one or more of the capsid proteins that package the vector, in which case at least one of the three capsid proteins can be from AAV1, AAV2, AAV3, AAV4, AAV5, AAV6, AAV7, AAV8, AAV9, AAV10, AAV11, RhlO, Rh74 or AAV-2i8 or variant, such as capsid variants such as LK03, 4-1, and the like. AAV vectors, therefore, can include nucleic acid and/or protein sequences identical to nucleic acid acnd protein sequences characteristic for a particular serotype.

An “AAV ITR” or “AAV ITRs” refers to the art-recognized regions found at each end of the AAV genome which function together in cis as origins of DNA replication and as packaging signals for the virus. AAV ITRs, together with the AAV rep coding region, provide for the efficient excision and rescue from and integration of a nucleotide sequence interposed between two flanking ITRs into a mammalian cell genome.

The nucleotide sequences of AAV ITRs are known. An “AAV ITR” need not have the wild-type nucleotide sequence depicted, but may be altered, e.g., by the insertion, deletion, or substitution of nucleotides. Additionally, the AAV ITR can be derived from any of several AAV serotypes. Furthermore, 5′ and 3′ ITRs which flank a heterologous nucleic acid sequence in an AAV vector need not necessarily be identical or derived from the same AAV serotype or isolate, so long as they function as intended, for example, to allow for excision and rescue of the sequence of interest from a host cell genome or vector, and to allow integration of the heterologous sequence into the recipient cell genome.

AAV “empty capsids” as used herein do not contain a vector genome (hence, the term “empty”), in contrast to “genome containing capsids,” which contain an AAV vector genome. Empty capsids are virus-like particles in that they react with one or more antibodies that react with the intact virus, such as a genome containing AAV vector. AAV empty capsids are believed to bind to or react with antibodies against the AAV vectors, thereby functioning as a decoy to reduce inactivation of the AAV vector. Such a decoy acts to absorb antibodies directed against the AAV vector, thereby increasing or improving AAV vector transgene transduction of cells (introduction of the transgene), and in turn, increased cellular expression of the transcript and/or encoded protein. Empty capsids can be generated and purified to the desired quality, and their quantities can be determined. For example, empty capsid titer can be measured by spectrophotometry by optical density at 280 nm wavelength (based on Sommer et al., Mol. Ther. 2003 January; 7(1): 122-8). Empty-AAV or empty capsids are sometimes naturally found in AAV vector preparations. Such natural mixtures can be used as described herein or if desired, be manipulated to increase or decrease the amount of empty capsid and/or vector. For example, the amount of empty capsid can optionally be adjusted to an amount that would be expected to reduce the inhibitory effect of antibodies that react with an AAV vector that is intended to be used for vector-mediated gene transduction in the subject. The use of empty capsids is described in US Publication 2014/0336245. In embodiments, AAV empty capsids are formulated with rAAV vectors and/or administered to a subject. In embodiments, AAV empty capsids are formulated with less than or an equal amount of vector, for example, about 1.0 to 100-fold AAV vectors to AAV empty capsids, or about a 1: 1 ratio of AAV vectors to AAV empty capsids. In embodiments, AAV vectors are formulated with an excess of AAV empty capsids, such as greater than 1-fold AAV empty capsids to AAV vectors, or 1.0 to 100-fold AAV empty capsids to AAV vectors.

In embodiments, the pair of ITRs are derived or obtained from or comprise the ITR sequences from AAV1, AAV2, AAV3, AAV4, AAV5, AAV6, AAV7, AAV8, AAV9, AAV10, AAV11, AAV 12, LK01, LK02, LK03, AAV 4-1, AAV-2i8 ITRs, or a combination thereof.

In embodiments, the one or more AAV capsid proteins are derived or obtained from or comprise one or more capsid protein sequences from AAV1, AAV2, AAV3, AAV4, AAV5, AAV6, AAV7, AAV8, AAV9, AAV10, AAV11, AAV 12, LK01, LK02, LK03, AAV 4-1, AAV-2i8 ITRs, or a combination thereof.

In embodiments, the rAAV vector further comprises one or more of an intron, an expression control element, a filler polynucleotide sequence and/or poly A signal, or a combination thereof.

rAAV vectors, rAAV particles, and pharmaceutical compositions thereof can be packaged in single or multiple unit dosage forms for ease of administration and uniformity of dosage. A “unit dose” or a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce the desired effect (e.g., prophylactic or therapeutic effect). Unit dosage forms can be within, for example, ampules and vials, which can include a liquid composition or a composition in a freeze-dried or lyophilized state; a sterile liquid carrier, for example, can be added prior to administration or delivery in vivo. Individual unit dosage forms can be included in unit-dose or multi-dose kits or containers.

In embodiments, the rAAV particles are administered at a dose in a range from about 1×10⁸ to 1×10¹⁰, 1×10¹⁰ to 1×10¹¹, 1×10¹¹ to 1×10¹², 1×10¹² to 1×10¹³, or 1×10¹³ to 1×10¹⁴ vector genomes per kilogram (vg/kg) bodyweight of the subject. In embodiments, the rAAV particles are administered at a dose of less than 1×10¹⁰ vector genomes per kilogram (vg/kg) of the subject.

In embodiments, the rAAV particles are administered at a dose of about 5×10¹¹ vector genomes per kilogram (vg/kg) of the subject. In embodiments, the rAAV particles administered are at least 1×10¹⁰ vector genomes (vg) per kilogram (vg/kg) of the bodyweight of the subject, or between about 1×10¹⁰ to 1×10¹¹ vg/kg of the bodyweight of the subject, or between about 1×10¹¹ to 1×10¹² vg/kg (e.g., about 1×10¹¹ to 2×10¹¹ vg/kg or about 2×10¹¹ to 3×10¹¹ vg/kg or about 3×10¹¹ to 4×10¹¹ vg/kg or about 4×10¹¹ to 5×10¹¹vg/kg or about 5×10¹¹ to 6×10¹¹ vg/kg or about 6×10¹¹ to 7×10¹¹ vg/kg or about 7×10¹¹ to 8×10¹¹ vg/kg or about 8×10¹¹ to 9×10¹¹ vg/kg or about 9×10¹¹ to 1×10¹² vg/kg) of the bodyweight of the subject, or between about 1×10¹⁰ to 1×10¹² vg/kg of the bodyweight of the subject, to achieve a desired therapeutic effect.

In embodiments, the subject does not develop a substantial immune response against the rAAV particle.

In embodiments, the subject does not develop a substantial humoral immune response against the rAAV particle.

In embodiments, the subject does not develop a substantial immune response against the rAAV particle for at least 1, 2, 3, 4, 5, 6, 7, 8, 9, 10, 11, 12, 13, or 14 continuous days, weeks, or months.

In embodiments, the subject does not develop a detectable immune response against the rAAV particle.

In embodiments, the subject does not produce an amount of undesirable immune response against the therapeutic protein and/or the rAAV particle that blocks the therapeutic effect of the therapeutic protein in the subject for at least 1, 2, 3, 4, 5, 6, 7, 8, 9, 10, 11, 12, 13 or 14 continuous days, weeks, or months.

More information about AAV vectors, production of vectors, pharmaceutical compositions including AAV vectors, and methods thereof can be found in U.S. Pat. No. 9,433,688, which is incorporated by reference in its entirety.

In embodiments, the viral vector is an rAAV particle comprising an AAV capsid protein and a vector comprising the nucleic acid encoding an antigen binding molecule inserted between a pair of AAV inverted terminal repeats (ITRs) in a manner effective to infect the cells, organ, or tissue of the peripheral blood system in the subject such that the cells, organ or tissue express antigen binding molecule.

In embodiments, administering the viral vector to a subject comprises infusion or injection of the viral vector into the systemic circulation of the subject. In embodiments, the administering comprises intravenous, intra-arterial infusion, or injection into the systemic circulation of the subject.

In embodiments, the AAV is used to deliver an antigen binding molecule to a subject. The antigen binding molecule is a CAR that comprises an extracellular domain, a transmembrane domain, and an intracellular domain, and the extracellular domain binds an antigen.

In embodiments, the intracellular domain comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the intracellular domain comprises CD3 zeta as the signaling region or primary signaling region.

In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the antigen binding molecule is a T Cell Receptor (TCR). In embodiments, the TCR is a modified TCR. In embodiments, the TCR is derived from spontaneously occurring tumor-specific T cells in patients. In embodiments, the TCR binds to a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains. In embodiments, a T cell clone that expresses a TCR with a high affinity for the target antigen can be isolated. In embodiments, tumor-infiltrating lymphocytes (TILs) or peripheral blood mononuclear cells (PBMCs) can be cultured in the presence of antigen-presenting cells (APCs) pulsed with a peptide representing an epitope known to elicit a dominant T cell response when presented in the context of a defined HLA allele. High-affinity clones can be then selected based on MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle, such as a gammaretrovirus or lentivirus, can be then generated and tested for functionality (antigen specificity and functional avidity) and used to produce a clinical lot of the vector. An aliquot of the final product can be used to transduce the target T cell population (generally purified from patient PBMCs), which is expanded before infusion into the patient.

In embodiments, the binding element of the CAR can include any antigen binding moiety that, when bound to its cognate antigen, affects a tumor cell such that the tumor cell fails to grow or is promoted to die or diminish.

The nucleic acid sequences encoding the desired molecules, such as CAR molecules, can be obtained using recombinant methods known in the art, such as, for example, by screening libraries from cells expressing the gene, by deriving the gene from a vector known to include the same, or by isolating directly from cells and tissues containing the same, using standard techniques. Alternatively, the gene of interest can be produced synthetically rather than cloned.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for the replication and integration of eukaryotes. Typical cloning vectors contain transcription and translation terminators, initiation sequences, and promoters useful for regulation of the expression of the desired nucleic acid sequence.

Additional information related to the expression of synthetic nucleic acids encoding CARs and gene transfer into mammalian cells is provided in U.S. Pat. No. 8,906,682, incorporated by reference in its entirety.

The embodiments of the present disclosure further relate to vectors in which a DNA encoding a desired molecule of the present disclosure can be inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Pharmaceutical compositions of the present disclosure can be administered in a manner appropriate to the disease to be treated (or prevented). The quantity and frequency of administration will be determined by such factors as the condition of the patient, and the type and severity of the patient's disease, although appropriate dosages can be determined by clinical trials. As an example, pharmaceutical compositions disclosed herein include nucleic acids encoding CAR or vectors described herein and a pharmaceutically acceptable carrier.

The term “pharmaceutically acceptable” means approved by a regulatory agency of the U.S. Federal or a state government or the EMA (European Medicines Agency) or listed in the U.S. Pharmacopeia (United States Pharmacopeia-33/National Formulary-28 Reissue, published by the United States Pharmacopeial Convention, Inc., Rockville Md., publication date: April 2010) or other generally recognized pharmacopeia for use in animals, and more particularly in humans.

The term “carrier” refers to a diluent, adjuvant (e.g., Freund's adjuvant (complete and incomplete)), excipient, or vehicle with which the therapeutic is administered. Pharmaceutical carriers can be sterile liquids, such as water and oils, including those of petroleum, animal, vegetable, or synthetic origins, such as peanut oil, soybean oil, mineral oil, sesame oil, and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. For the use of (further) excipients and their use see also “Handbook of Pharmaceutical Excipients”, fifth edition, R. C. Rowe, P. J. Seskey and S. C. Owen, Pharmaceutical Press, London, Chicago.

The administration of the pharmaceutical compositions described herein can be carried out in any convenient manner, including by aerosol inhalation, injection, ingestion, transfusion, implantation, or transplantation. The compositions described herein can be administered to a patient subcutaneously, intradermally, intratumorally, intranodally, intramedullary, intramuscularly, by intravenous (i. v.) injection, or intraperitoneally. In embodiments, the T cell compositions of the present disclosure are administered to a patient by intradermal or subcutaneous injection. In another embodiment, the T cell compositions of the present disclosure are preferably administered by i.v. injection. The compositions of T cells can be injected directly into a tumor, lymph node, or site of infection. In embodiments of the present disclosure, cells activated and expanded using the methods described herein, or other methods known in the art where T cells are expanded to therapeutic levels, are administered to a patient in conjunction with (e.g., before, simultaneously, or following) any number of relevant treatment modalities, including but not limited to treatment with agents such as antiviral therapy, cidofovir, and interleukin-2, Cytarabine (also known as ARA-C) or natalizumab treatment for MS patients or efalizumab treatment for psoriasis patients or other treatments for PML patients. In embodiments, the T cells of the present disclosure can be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludarabine, cyclosporin, FK506, rapamycin, mycophenolic acid, steroids, FR901228, cytokines, and irradiation. These drugs inhibit either the calcium-dependent phosphatase calcineurin (cyclosporine and FK506) or inhibit the p70S6 kinase that is important for growth factor-induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously, or following) bone marrow transplantation, T cell ablative therapy using either chemotherapy agents such as fludarabine, external-beam radiation therapy (XRT), cyclophosphamide, or antibodies such as OKT3 or CAMPATH. In embodiments, the cell compositions of the present disclosure are administered following B-cell ablative therapy, such as agents that react with CD20, e.g., Rituxan. For example, subjects can undergo standard treatment with high dose chemotherapy followed by peripheral blood stem cell transplantation. In embodiments, following the transplant, subjects receive an infusion of the expanded immune cells of the present disclosure. In other embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician, depending on various factors.

When “an immunologically effective amount,” “an anti-tumor effective amount,” “a tumor-inhibiting effective amount,” “therapeutic amount,” or “effective amount” is indicated, the precise amount of the compositions of the present disclosure to be administered can be determined by a physician with consideration of individual differences in age, weight, tumor size, the extent of infection or metastasis, and condition of the patient (subject). It can be stated that a pharmaceutical composition comprising the T cells described herein can be administered at a dosage of 10⁴ to 10⁹ cells/kg body weight, preferably 10⁵ to 10⁶ cells/kg body weight, including all integer values within those ranges. T cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art by monitoring the patient for signs of disease and adjusting the treatment accordingly. In embodiments, it can be desired to administer activated T cells to a subject and then subsequently redraw the blood (or have apheresis performed), collect the activated and expanded T cells, and reinfuse the patient with these activated and expanded T cells. This process can be carried out multiple times every few weeks. In embodiments, T cells can be activated from blood draws of from 10 cc to 400 cc. In embodiments, T cells are activated from blood draws of 20 cc, 30 cc, 40 cc, 50 cc, 60 cc, 70 cc, 80 cc, 90 cc, or 100 cc. Not to be bound by theory, certain populations of T cells can be selected using this multiple blood draw/multiple reinfusion protocols.

Additional information on the methods of cancer treatment using engineered or modified T cells and sequences related to PAP CAR is provided in U.S. Pat. No. 8,906,682 and PCT Publication Nos. WO22150831 and WO20047306, incorporated by reference in its entirety.

Embodiments relate to an in vitro method for preparing modified cells. The method can include obtaining a sample of cells from the subject. For example, the sample can include T cells or T cell progenitors. The method can further include transfecting or transducing the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

In embodiments, the nucleic acid sequence encodes a humanized CD19 CAR described in PCT Publication No: WO2018126369, which is incorporated by its entirety.

In embodiments, the nucleic acid sequence encodes a CAR targeting an antigen of a non-essential tissue described in PCT Publication No: WO2018064921, which is incorporated herein by reference in its entirety.

In embodiments, the nucleic acid sequence encodes a CAR and a therapeutic agent described in PCT Publication No: WO2020106843, which is incorporated herein by reference in its entirety.

In embodiments, the nucleic acid sequence encodes one or more components of CoupledCAR® described in PCT Publication No: WO2020146743, which is incorporated herein by reference in its entirety.

In embodiments, the nucleic acid sequence encodes a modified immune checkpoint molecule (e.g., PD-1) as described in U.S. Pat. No. 9,572,837, which is incorporated herein by reference in its entirety. An immune checkpoint molecule refers to a molecule that is associated with T cells and regulates T cell response. In embodiments, the immune checkpoint molecule is selected from the group consisting of PD-1, cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD 160.

Embodiments describe a chimeric antigen receptor (CAR)-modified T cell for use in cancer immunotherapy, the CAR comprising: a signal peptide; a fully human single-chain variable fragment (scFv) that specifically binds transmembrane prostatic acid phosphatase (PAP); a transmembrane domain; a costimulatory signaling domain; and a CD3ζ signaling domain.

In embodiments, the signal peptide comprises a CD8a signal peptide. In embodiments, the scFv comprises the amino acid sequence of clone S5D1, which specifically binds to an epitope within the extracellular domain of human transmembrane PAP with nanomolar affinity. In embodiments, the scFv is humanized to reduce immunogenicity in a clinical setting.

In embodiments, the transmembrane domain comprises a CD8a transmembrane sequence. In embodiments, the costimulatory signaling domain comprises a 4-1BB intracellular domain. In embodiments, the CD3ζ signaling domain is derived from the human CD247 gene and provides primary activation signaling upon CAR engagement with antigen.

In embodiments, the CAR is introduced into T cells using a viral vector, optionally a lentiviral or retroviral vector. In embodiments, the CAR is co-expressed with a marker such as green fluorescent protein (GFP) via a 2A peptide linker or internal ribosome entry site (IRES). In some embodiments, the T cells expressing the CAR are expanded ex vivo using a protocol including anti-CD3/CD28 stimulation and interleukin-2.

In embodiments, the CAR-T cells exhibit specific cytotoxicity against tumor cells expressing PAP and show no significant cytotoxicity against PAP-negative control cells. In embodiments, the CAR-T cells produce effector cytokines, including IFN-γ and TNF-α, upon recognition of PAP-positive tumor targets.

In further embodiments, the CAR-T cells are administered to a subject having prostate cancer, including metastatic or castration-resistant forms. In embodiments, the CAR-T cells are infused intravenously at a dose ranging from 1×10⁶ to 1×10⁸ cells per kg body weight. In embodiments, the treatment may be combined with lymphodepleting chemotherapy, checkpoint inhibitors, or other immunomodulatory agents.

Embodiments describe a system and method for antigen-independent expansion of a solid tumor-targeting chimeric antigen receptor T (CAR-T) cell population in a subject, the method comprising: co-administering a first CAR-T cell population that targets a solid tumor antigen, a second CAR-T cell population that targets an immune cell antigen such as CD19, and an auxiliary cell population comprising immune cells such as B cells expressing the immune cell antigen.

In embodiments, the first CAR-T cell population comprises T cells engineered to express a CAR targeting transmembrane prostatic acid phosphatase (PAP). In embodiments, the second CAR-T cell population comprises T cells engineered to express a CAR targeting CD19, optionally incorporating a CD8a transmembrane domain, a 4-1BB costimulatory domain, and a CD3ζ activation domain.

In embodiments, the auxiliary cell population comprises autologous B cells expressing CD19, obtained from the subject's peripheral blood via leukapheresis or magnetic bead-based selection. In embodiments, the auxiliary B cells are administered in multiple doses following infusion of the CAR-T cell populations, such as on days 3, 6, and 9 post-infusion.

In embodiments, the expansion of the first CAR-T cell population occurs in the absence of antigen engagement with the target solid tumor antigen. In embodiments, the expansion is mediated by costimulatory signals comprising CD80 and CD86 expressed on B cells, and cytokine signals comprising IL-2, IL-15, IL-7, and/or chemokines such as CXCL9 secreted by B cells or CD19 CAR-T cells.

In embodiments, the expanded CAR-T cells exhibit a memory-like phenotype, including upregulation of TCF7, IL7R, and CCR7, and downregulation of exhaustion markers such as PD-1, LAG-3, and NR4A family transcription factors. In embodiments, the CoupledCAR system enables enhanced persistence and antitumor activity of the first CAR-T population in vivo.

In further embodiments, the method reduces dependence on tumor-localized antigen stimulation and minimizes TCR/CD3ζ signaling in the first CAR-T cell population, thereby decreasing the risk of CAR-T-associated toxicities such as cytokine release syndrome (CRS) and immune effector cell-associated neurotoxicity syndrome (ICANS).

In embodiments, the second CAR-T cell population and B cells may be modified, substituted, or adapted to target alternative circulating leukocyte antigens such as CD20, CD22, or BCMA to replicate the antigen-independent expansion effect. In embodiments, the CoupledCAR system may be applied to TCR-T cells, tumor-infiltrating lymphocytes (TILs), or other adoptive cell therapies to enhance functional expansion without TCR engagement.

Embodiments describe a method for reducing adverse events associated with CAR-T cell therapy, the method comprising: administering to a subject a first CAR-T cell population that targets a solid tumor antigen, a second CAR-T cell population that targets an immune cell antigen such as CD19, and an auxiliary immune cell population such as B cells expressing the immune cell antigen; wherein the first CAR-T cell population expands in vivo without activation of T cell receptor (TCR) or CD3ζ signaling.

In embodiments, the first CAR-T cell population comprises T cells expressing a chimeric antigen receptor specific for transmembrane prostatic acid phosphatase (PAP). In embodiments, the second CAR-T cell population expresses a CD19-specific CAR comprising a CD8a transmembrane domain, a 4-1BB costimulatory domain, and a CD3ζ activation domain. In embodiments, the auxiliary B cells are autologous and administered sequentially on days 3, 6, and 9 post-infusion.

In embodiments, the expansion of the first CAR-T cell population is driven by costimulatory signals (e.g., CD80, CD86) and cytokines or chemokines (e.g., IL-2, IL-15, CXCL9) produced by the interaction between CD19 CAR-T cells and B cells. In embodiments, the system is configured such that the first CAR-T cell population is not exposed to its cognate tumor antigen in vivo during expansion, thereby preventing CD3ζ-mediated activation.

In embodiments, the resulting CAR-T cell phenotype is characterized by a memory-like transcriptional profile, including high expression of TCF7, IL7R, CCR7, and SELL, and reduced expression of exhaustion-associated genes including PDCD1, LAG3, and the NR4A family (NR4A1, NR4A2, NR4A3). In embodiments, this transcriptional program is confirmed using single-cell RNA sequencing and/or flow cytometry analysis of surface markers.

In embodiments, the method results in decreased levels of systemic inflammatory cytokines associated with CAR-T toxicities, including IL-6, IFN-7, and TNF-α, and improved immune effector cell-associated neurotoxicity syndrome (ICANS) scores as assessed using standard metrics such as the ICE score.

In embodiments, the method reduces the incidence or severity of cytokine release syndrome (CRS) and ICANS compared to conventional CAR-T therapies dependent on antigen-driven expansion. In embodiments, this AE mitigation strategy improves CAR-T persistence, tumor infiltration, and long-term antitumor efficacy.

In further embodiments, the approach is compatible with solid tumor-directed CAR-T cell products beyond PAP, and may be adapted to CD20-, BCMA-, or CD22-based expansion modules. In embodiments, the AE mitigation effect is intrinsic to the programmed expansion conditions and does not require incorporation of exogenous suicide genes or pharmacologic control switches.

Embodiments describe a method for identifying and optimizing single-chain variable fragments (scFvs) that specifically bind transmembrane prostatic acid phosphatase (tmPAP), the method comprising: generating a human antibody library; screening the library against recombinant tmPAP protein; and selecting clones with high binding affinity and specificity.

In embodiments, the recombinant tmPAP comprises the extracellular domain of human PAP with a purification tag and is expressed in mammalian cells to preserve native conformation. In embodiments, ELISA, flow cytometry, and BIAcore (surface plasmon resonance) assays are used to identify high-affinity binders, including scFv clone S5D1, with a KD in the subnanomolar to low nanomolar range.

In embodiments, the selected scFv is humanized to reduce immunogenicity and cloned into a chimeric antigen receptor (CAR) construct comprising a CD8a transmembrane domain, a 4-1BB costimulatory domain, and a CD3ζ activation domain. In embodiments, the scFv sequence is fused to a signal peptide (e.g., CD8α) and optionally co-expressed with a fluorescent reporter such as GFP via a 2A peptide or IRES.

In embodiments, the functional activity of the resulting CAR-T cells is evaluated using in vitro cytotoxicity assays against PAP-positive target cells and in vivo using a PC3-tmPAP xenograft model. In embodiments, CAR-T cells are infused intravenously and tumor volume, persistence, and cytokine release are monitored longitudinally to assess therapeutic efficacy.

Embodiments describe a coculture expansion method for antigen-independent proliferation of solid tumor-targeting CAR-T cells, the method comprising: combining a first CAR-T cell population specific for a solid tumor antigen, a second CAR-T cell population specific for a lymphocyte surface antigen such as CD19, and an auxiliary population of lymphocytes expressing the lymphocyte surface antigen, at a predetermined effector-to-target ratio.

In embodiments, the CAR-T cell populations and B cells are co-cultured ex vivo under controlled conditions, and expansion of the first CAR-T cell population is monitored using CellTrace dye dilution, flow cytometry, and cytokine profiling. In embodiments, IL-2, IL-15, and CXCL9 levels are quantified to assess cytokine contribution to expansion dynamics.

In embodiments, the CD19 CAR-T cells may be substituted with alternative immune cell-targeting CARs such as anti-CD20, anti-CD22, or anti-BCMA, allowing for modular system adaptation. In embodiments, the cell ratio between CD19 CAR-T cells and B cells is titrated (e.g., 1:1 to 1:10) to optimize expansion outcomes.

In embodiments, the coculture system enables functional programming of the solid tumor CAR-T cells without antigen-dependent CD3ζ signaling, preserving central memory and stem-like phenotypes. In embodiments, the coculture process is implemented in a closed or semi-automated bioreactor platform for clinical-scale manufacturing.

Embodiments describe a method for characterizing the phenotype and exhaustion status of expanded CAR-T cells, the method comprising: performing single-cell RNA sequencing (scRNA-seq) on T cells harvested after in vivo or ex vivo expansion; and analyzing gene expression profiles indicative of memory differentiation, trafficking, and exhaustion.

In embodiments, PAP CAR-T cells expanded using the CoupledCAR system are subjected to scRNA-seq using 10× Genomics technology. In embodiments, bioinformatic analysis identifies increased expression of memory markers such as TCF7, IL7R, CCR7, and SELL, and decreased expression of exhaustion markers including PDCD1, LAG3, NR4A1, NR4A2, and NR4A3.

In embodiments, the method further comprises applying the CellChat computational pipeline to infer ligand-receptor interactions between CD19 CAR-T cells, B cells, and PAP CAR-T cells, thereby identifying the source and timing of CD80/CD86-mediated costimulation and cytokine support.

In embodiments, peripheral blood samples from treated subjects are analyzed to quantify the impact of B cell presence on systemic inflammatory cytokine levels and B cell counts, confirming that CD19 CAR-T activity selectively modulates auxiliary B cells without depleting critical immune compartments. These data collectively support the built-in safety and low-toxicity profile of the CoupledCAR-mediated expansion strategy,

Embodiments describe a chimeric antigen receptor-modified T cell (CAR-T cell) for use in treating prostate cancer, the CAR comprising: a human-derived single-chain variable fragment (scFv) that binds specifically to transmembrane prostatic acid phosphatase (PAP); a signal peptide; a transmembrane domain; a costimulatory signaling domain; and a CD3ζ activation domain.

In embodiments, the scFv is derived from clone S5D1, selected via screening of human antibody libraries against a recombinant PAP extracellular domain. In embodiments, the scFv is humanized to minimize immunogenicity and exhibits nanomolar or subnanomolar affinity, as confirmed by surface plasmon resonance.

In embodiments, the CAR comprises a CD8a signal peptide, a CD8a transmembrane domain, a 4-1BB (CD137) costimulatory domain, and a CD3ζ signaling domain. In embodiments, the CAR is introduced into autologous human T cells using a lentiviral or retroviral vector and optionally co-expressed with a GFP reporter for tracking.

In embodiments, the CAR-T cells exhibit antigen-specific cytotoxicity against PAP-positive tumor cells in vitro and in vivo. In embodiments, the CAR-T cells are administered to subjects with advanced or castration-resistant prostate cancer, optionally following lymphodepletion. In embodiments, tumor regression, CAR-T expansion, and cytokine secretion are monitored as clinical endpoints.

Embodiments describe a system for expanding a solid tumor-targeting CAR-T cell population in vivo in the absence of antigen engagement, the system comprising: a first CAR-T cell population specific for a solid tumor antigen; a second CAR-T cell population specific for a circulating lymphocyte antigen; and an auxiliary cell population expressing said lymphocyte antigen.

In embodiments, the first CAR-T cells target PAP; the second CAR-T cells target CD19; and the auxiliary cells are autologous B cells. In embodiments, the B cells provide costimulatory signals (CD80, CD86) and cytokines (IL-2, IL-15, CXCL9) upon engagement by CD19 CAR-T cells.

In embodiments, the components are co-administered to a subject with a solid tumor. In embodiments, B cells are infused in repeated doses post-T cell infusion. In embodiments, the first CAR-T cells undergo expansion and differentiation into memory phenotypes without activation of CD3ζ signaling.

In embodiments, the second CAR-T cell population may be substituted with other CARs (e.g., CD20, CD22, BCMA) targeting alternate leukocyte antigens, enabling a modular platform. In embodiments, the system is used in both therapeutic and ex vivo manufacturing settings for consistent, non-antigen-dependent T cell expansion.

Embodiments describe a method for reducing adverse events in CAR-T cell therapy, the method comprising: administering to a subject a first CAR-T cell population targeting a solid tumor antigen; a second CAR-T cell population targeting a leukocyte antigen; and an auxiliary cell population expressing said antigen, wherein the expansion of the first CAR-T cell population occurs without activation of CD3ζ signaling.

In embodiments, the expansion is driven by costimulatory and cytokine/chemokine signals delivered by interaction between CD19 CAR-T cells and B cells. In embodiments, this antigen-independent expansion prevents excessive immune activation and reduces the incidence and severity of cytokine release syndrome (CRS) and immune effector cell-associated neurotoxicity syndrome (ICANS).

In embodiments, the expanded CAR-T cells exhibit a memory-like phenotype, with increased expression of IL7R, TCF7, and CCR7 and reduced expression of PD-1, LAG3, and NR4A transcription factors. In embodiments, the phenotype is validated using single-cell RNA sequencing, flow cytometry, and CellChat-based ligand-receptor analysis.

In embodiments, the method reduces systemic cytokine levels (e.g., IL-6, IFN-γ) and preserves functional T cell persistence. In embodiments, the method is implemented during initial infusion, as a preconditioning expansion step, or during therapeutic manufacturing. In further embodiments, the strategy applies to TCR-T or TIL therapies to improve safety and durability.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples but rather should be construed to encompass any and all variations which become evident as a result of the teaching provided herein.

### EXEMPLARY EMBODIMENTS

The following are exemplary embodiments:
1. A method of expanding a solid tumor-targeting chimeric antigen receptor T (CAR-T) cell population in a subject, the method comprising:
co-administering to the subject:
a) a first CAR-T cell population comprising T cells expressing a chimeric antigen receptor that specifically binds a solid tumor antigen;
b) a second CAR-T cell population comprising T cells expressing a chimeric antigen receptor that specifically binds CD19; and
c) B cells expressing CD19;
wherein the expansion of the first CAR-T cell population occurs in vivo in the absence of exposure to its cognate tumor antigen and is mediated by costimulatory signals and cytokine signals derived from the interaction of the second CAR-T cell population with the B cells.
2. The method of embodiment 1, wherein the solid tumor antigen is transmembrane prostatic acid phosphatase (tmPAP).
3. The method of embodiment 1 or 2, wherein the chimeric antigen receptor of the first CAR-T cell population comprises a single-chain variable fragment (scFv) derived from a human antibody that binds specifically to tmPAP.
4. The method of any one of embodiments 1-3, wherein the chimeric antigen receptor of the second CAR-T cell population comprises a 4-1BB costimulatory domain and a CD3ζ signaling domain.
5. The method of any one of embodiments 1-4, wherein the B cells are administered to the subject at multiple time points following the administration of the first and second CAR-T cell populations.
6. The method of any one of embodiments 1-5, wherein the costimulatory signals comprise CD80 and CD86 expressed on the surface of B cells.
7. The method any one of embodiments 1-6, wherein the cytokine signals comprise at least one of IL-2, IL-15, IL-3, IL-4, or CXCL9 secreted by the B cells or the second CAR-T cell population.
8. The method of any one of embodiments 1-7, wherein the expansion of the first CAR-T cell population results in a T cell phenotype characterized by increased expression of CCR7, IL7R, and TCF7, and decreased expression of PD-1 and LAG-3.
9. The method of any one of embodiments 1-8, wherein the first CAR-T cell population retains a central memory (TCM) or stem-cell memory (T_SCM) phenotype after expansion.
10. The method of any one of embodiments 1-9, wherein the B cells are obtained from the subject's peripheral blood and isolated using a B cell selection kit.
11. The method of any one of embodiments 1-10, wherein the subject is a human diagnosed with metastatic or treatment-refractory prostate cancer.
12. The method of any one of embodiments 1-11, wherein the expansion of the first CAR-T cell population is not accompanied by activation of T cell receptor (TCR) or CD3ζ signaling in the first CAR-T cell population.
13. The method of any one of embodiments 1-12, wherein the second CAR-T cell population and the B cells are administered at an effector-to-target ratio of between 1:1 and 1:10.
14. The method of any one of embodiments 1-13, wherein the expansion of the first CAR-T cell population is associated with increased antitumor efficacy in a solid tumor xenograft model.
15. The method of any one of embodiments 1-14, wherein the method further comprises measuring cytokine levels in the peripheral blood of the subject after treatment to monitor CAR-T cell expansion.
16 A method for reducing treatment-associated adverse events in a subject receiving chimeric antigen receptor T (CAR-T) cell therapy, the method comprising:
administering to the subject a first population of CAR-T cells that target a solid tumor antigen;
administering to the subject a second population of CAR-T cells that target CD19; and co-administering B cells expressing CD19;
wherein the expansion of the first population of CAR-T cells occurs in vivo without stimulation of T cell receptor (TCR) or CD3ζ signaling in the first population, and
wherein the first population of CAR-T cells exhibits a memory-like phenotype and reduced expression of exhaustion markers, thereby reducing the incidence or severity of cytokine release syndrome or immune effector cell-associated neurotoxicity in the subject.
17. The method of embodiment 16, wherein the first population of CAR-T cells expresses a chimeric antigen receptor comprising a fully human scFv that binds transmembrane prostatic acid phosphatase (tmPAP).
18. The method of embodiment 16 or 17, wherein the memory-like phenotype comprises expression of at least one of IL7R, TCF7, CCR7, or LEF1.
19. The method of any one of embodiments 16-18, wherein the reduced expression of exhaustion markers comprises downregulation of at least one of PDCD1, LAG3, NR4A1, NR4A2, or NR4A3.
20. The method of any one of embodiments 16-19, wherein the expansion of the first CAR-T cells is mediated by CD80/CD86 costimulatory signaling and cytokines selected from IL-2, IL-15, and CXCL9.
21. The method of any one of embodiments 16-20, wherein the subject is monitored for cytokine release syndrome by measuring serum levels of IL-6, IFN-γ, and TNF-α within 24 hours post-administration.
22. The method of any one of embodiments 16-21, wherein the CD19-targeting CAR-T cells comprise a 4-1BB costimulatory domain and a CD3ζ signaling domain.
23. The method of any one of embodiments 16-22, wherein the B cells are autologous and are administered on at least two separate days post-infusion of the CAR-T cells.
24. The method of any one of embodiments 16-23, wherein the first CAR-T cells maintain a TCF7⁺ CCR7⁺ CD45RA⁺ phenotype for at least five days post-expansion in the subject.
25. The method of any one of embodiments 16-24, wherein the method results in reduced infiltration of inflammatory myeloid cells into the tumor microenvironment compared to control CAR-T therapy lacking CD19 CAR-T and B cell co-administration.
26. The method of any one of embodiments 16-25, wherein the method further comprises administering a pre-conditioning lymphodepletion regimen selected from fludarabine, cyclophosphamide, or a combination thereof.
27. The method of any one of embodiments 16-26, wherein the incidence of neurotoxicity is measured using ICE (Immune effector Cell-Associated Encephalopathy) scores and is at least 50% lower than that observed with CAR-T therapy lacking the second CAR-T cell and B cell populations.
28. A system for expanding a solid tumor-targeting chimeric antigen receptor T (CAR-T) cell population in a subject, the system comprising:
a) a first CAR-T cell population comprising T cells engineered to express a chimeric antigen receptor that specifically binds a solid tumor antigen;
b) a second CAR-T cell population comprising T cells engineered to express a chimeric antigen receptor that specifically binds a surface antigen expressed on circulating lymphocytes; and
c) an auxiliary cell population comprising lymphocytes expressing the surface antigen bound by the second CAR-T cell population;
wherein the expansion of the first CAR-T cell population in vivo is driven by costimulatory signals and cytokine signals resulting from the interaction of the second CAR-T cell population with the auxiliary cell population, and
wherein the expansion occurs in the absence of cognate antigen engagement by the first CAR-T cell population.
29. The system of embodiment 28, wherein the surface antigen is CD19, and the auxiliary cell population comprises autologous B cells expressing CD19.
30. The system of embodiment 28 or 29, wherein the first CAR-T cell population comprises a CAR with a fully human single-chain variable fragment (scFv) that binds transmembrane prostatic acid phosphatase (tmPAP).
31. The system of any one of embodiments 28-30, wherein the costimulatory signals comprise CD80 and CD86 expressed on the auxiliary cell population.
32. The system of any one of embodiments 28-31, wherein the cytokine signals comprise at least one of IL-2, IL-15, IL-7, or CXCL9.
33. The system of any one of embodiments 28-32, wherein the second CAR-T cell population comprises a CAR with a 4-1BB costimulatory domain and a CD3ζ signaling domain.
34. The system of any one of embodiments 28-33, wherein the auxiliary cell population is administered to the subject in multiple doses post-infusion of the CAR-T cells.
35. The system of any one of embodiments 28-34, wherein the expansion of the first CAR-T cell population results in a central memory phenotype characterized by expression of TCF7, CCR7, and IL7R.
36. The system of any one of embodiments 28-35, wherein the expansion of the first CAR-T cell population occurs without activation of CD3ζ signaling in the first CAR-T cells.
37. The system of any one of embodiments 28-36, wherein the first CAR-T cells demonstrate reduced expression of exhaustion markers selected from PD-1, LAG-3, NR4A1, or NR4A3 following expansion in vivo.
38. The system of any one of embodiments 28-37, wherein the solid tumor antigen is selected from the group consisting of PAP, GCC, CEA, MUC1, mesothelin, and HER2.
39. The system of any one of embodiments 28-38, wherein the system is used for treating a solid tumor selected from prostate cancer, colorectal cancer, pancreatic cancer, or ovarian cancer.
40. The system of any one of embodiments 28-39, wherein the first and second CAR-T cell populations are cryopreserved and thawed immediately prior to administration.
41. The system of any one of embodiments 28-40, wherein the auxiliary cell population is expanded ex vivo prior to administration using a B cell stimulation cocktail comprising CD40L, IL-4, and CpG oligonucleotides.
42. The system of any one of embodiments 28-41, wherein the system further comprises a closed-loop infusion apparatus configured to deliver the three cell populations sequentially or concurrently to the subject.

### EXAMPLES

Lentiviral vectors that encode individual CAR molecules were generated and transfected with T cells, which are described below. Techniques related to cell cultures, construction of cytotoxic T lymphocyte assay can be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in its entirety.

Background The challenges that remain in the treatment of solid tumors with chimeric antigen receptor (CAR)-T cells include limited solid tumor-specific targets and poor CAR-T cell expansion and function due to limited availability of solid tumor antigens outside the tumor microenvironment. Prostate cancer is the second most common cancer among men worldwide. Current CAR-T therapies for prostate cancer lack specific targets, posing safety risks. To overcome these problems, we identified prostatic acid phosphatase (PAP) as a feasible CAR-T target for prostate cancer and developed CoupledCAR, a novel approach for expanding tumor-targeting CAR-T cells without tumor antigens.

Methods We analyzed the expression of PAP from The Cancer Genome Atlas (TCGA) database and validated its expression in normal and cancer tissues through immunohistochemistry (IHC) staining. To generate anti-PAP specific antibodies, we screened the human single-chain antibody library using tmPAP-His antigen and selected antibodies based on their binding ability and specificity. We constructed PAP-targeted CAR and evaluated their antitumor efficacy both in vitro and in vivo. We validated the function of PAP CoupledCAR in both in vitro and in vivo experiments, and further analyzed its mechanism using single-cell RNA sequencing (scRNA-Seq).

Results PAP was specifically expressed in prostate epithelial and prostate cancer cells, with no expression in other tissues. Seven single-chain variable fragments (scFvs) were screened from the human single-chain antibody library, with S5D1 showing the highest binding ability for the PAP. PAP CAR-T cells demonstrated strong antitumor efficacy both in vitro and in vivo. Furthermore, the CoupledCAR system significantly expanded PAP CAR-T cells, promoting memory-like status, reducing exhaustion, and enhancing their antitumor efficacy. The scRNA-Seq demonstrated that the expansion of PAP CAR-T cells in the CoupledCAR system is mediated by costimulatory signals and cytokine signals, rather than T-cell receptor (TCR) signals.

Conclusions Our study is the first to demonstrate that PAP is a specific target for CAR-T therapy in prostate cancer, both in vitro and in vivo. We developed the CoupledCAR platform technology for solid tumor CAR-T cell therapy, enabling the expansion of tumor-targeting CAR-T cells without requiring tumor antigens and thereby enhancing their functionality against solid tumors.

Several targets, such as prostate-specific membrane antigen (PSMA), have been explored for CAR-T cell therapy in prostate cancer. However, these targets lack sufficient specificity, posing safety risks such as neurotoxicity, and their clinical efficacy remains limited. Moreover, insufficient in vivo expansion of CAR-T cells has also significantly limited the efficacy of CAR-T therapy in solid tumors.

This study is the first to identify and validate PAP as a specific target for CAR-T therapy in prostate cancer, and also the first to discover a fully humanized anti-PAP antibody with strong binding ability and specificity. Based on scFv form of this antibody, we validated the efficacy of CAR-T cells targeting the PAP antigen. Additionally, we developed a novel CoupledCAR approach that significantly expands PAP CAR-T cells and enhances their antitumor efficacy compared to conventional CAR-T cells.

Our preclinical data provided the foundation for clinical trials evaluating PAP-targeted CoupledCAR-T therapy in the treatment of advanced prostate cancer. And the CoupledCAR system shows potential for application in other solid tumors, offering a platform for the development of CAR-T therapies targeting other solid tumors.

Chimeric antigen receptor (CAR)-T cells have achieved marked clinical responses, including a high proportion of durable complete remissions, in patients with hematological malignancies.1 2 3 However, CAR-T therapy has shown limited efficacy in the treatment of solid tumors. One major challenge in the treatment of solid tumors is the lack of specific surface targets, which severely limits the application of CAR-T cells in solid tumors. 4 5 Prostate cancer is the second most common cancer among men worldwide, and patients with late-stage metastatic prostate cancer have a five-year survival rate of only 30%.6 7 Therefore, there is an urgent need to develop effective treatments for advanced prostate cancer. Currently, several targets, such as prostate-specific membrane antigen (PSMA), have been tested for CAR-T cell therapy in prostate cancer.8 However, since PSMA is also expressed in the central nervous system, it poses potential safety risks related to neurotoxicity.9

Another major challenge in the treatment of solid tumors is insufficient expansion of solid tumor-targeting CAR-T cells.10 11 In hematological malignancies, CAR-T cells can recognize tumor antigens in the bloodstream, such as CD19 or B-cell maturation antigen (BCMA), which allows for rapid expansion and significant antitumor effects.12 However, in patients with solid tumors, CAR-T cells often cannot effectively access tumor antigens shielded by the tumor microenvironment,13 14 resulting in a lack of expansion signals and poor CAR-T cell proliferation, which limits their antitumor activity.15

Here, we utilized a novel target screening approach and characterized prostatic acid phosphatase (PAP) as a feasible target for the treatment of prostate cancer with CAR-T cell therapy. An approach called CoupledCAR was also developed to expand solid tumor-targeting CAR-T cells without the need for solid tumor antigens, resulting in less exhaustion, better memory status and better killing ability than conventional CAR-T cell technology. Recently, GCC19CAR-T, based on the CoupledCAR technology, showed impressive efficacy in a clinical study targeting guanylate cyclase-C (GCC) in third-line metastatic colorectal cancer (mCRC).16 In this study, we further clarify the underlying mechanism of CoupledCAR and demonstrated that CoupledCAR functions as a platform technology, showing similar efficacy when applied to the PAP target.

Preparation of tmPAP Antigen and Screening of Anti-PAP Specific scFvs: The transmembrane PAP(tmPAP) gene (Sino Biological, Inc., HG10959-M) was cloned into the recombinant antigen expression vector PTSE-His (Beijing Bettermab Biotechnology Co., Ltd.). The constructed recombinant plasmid was transfected into HEK293 cells (Beijing Bettermab Biotechnology Co., Ltd.), and the recombinant tmPAP-His protein expressed by the HEK293 cells was purified using a HisTrap FF affinity chromatography column (GE Healthcare Bio-Sciences). The purified recombinant tmPAP-His protein was used as an antigen for screening a human single-chain antibody library (Beijing Bettermab Biotechnology Co., Ltd.). After three rounds of screening, 40 monoclonal single chain antibodies that specifically bind to the tmPAP antigen were obtained, from which seven distinct antibody sequences were identified and designated as S4C7, S5D1, S5F2, S9F5, S10E12, S12D4, and S12D9. These single-chain antibody fragments were inserted into phage vectors (Beijing Bettermab Biotechnology Co., Ltd) to generate monoclonal single-chain antibody phages, followed by antigen-binding analysis using the recombinant tmPAP-His protein.

Characterization of fully humanized PAP antibodies: The variable regions of the heavy and light chains of the selected scFv antibodies were cloned into the eukaryotic expression vectors pTSEG1n and pTSEK (Beijing Bettermab Biotechnology Co., Ltd), respectively, and transfected into HEK293 cells to produce fully humanized antibodies. The recombinant humanized antibodies were purified using a Protein A affinity chromatography column. TmPAP-His antigen was coated onto a plate, and after blocking, gradient-diluted purified recombinant humanized antibodies were added. Anti-human IgG-HRP secondary antibody (Beijing Bettermab Biotechnology Co., Ltd) was added for detection. The binding was quantified via a colorimetric reaction, and monoclonal antibodies that specifically bound to the tmPAP-His antigen were identified.

Binding ability and affinity analysis of monoclonal antibodies: In a flow cytometry-based assay, PC3-tmPAP cells were labeled separately with each of the seven fully recombinant humanized antibodies for 0.5 hours at 4 °C. After washing, the cells were stained with goat anti-human IgG-AF488 (Abcam, Ab96907) in stain buffer for 0.5 hours at 4 °C. Following two washes, the cells were analyzed on a CytoFlex flow cytometer (Beckman Coulter) through the FITC channel. The binding of PAP antibodies was determined by the mean fluorescence intensity of Alexa Fluor 488 on live PC3-tmPAP cells. In the affinity analysis experiment, the S5D1 antibody was coated onto a pre-treated chip, and different concentrations of recombinant human tmPAP protein were added as the mobile phase. Antibody affinity was measured using the BIAcore X-100 (GE Healthcare Bio-Sciences) system.

Healthy donors and cell lines: Peripheral blood samples were collected from healthy donors who provided informed consent. The human prostate cancer cell line PC3 was cultured in F12-K medium (Gibco, 21127-022) supplemented with 10% fetal bovine serum (FBS, Gibco, 16000-044). PC3 cells were transduced with a lentiviral vector expressing the tmPAP gene to generate PC3-tmPAP cells. The CD19-positive Nalm6 cell line was cultured under standard conditions (F12-K medium supplemented with 10% FBS). The human embryonic kidney cell line 293T (Thermo, A35347) was cultured in DMEM medium (Gibco, 11995-040) supplemented with 10% FBS.

Vector construction: First, the signal peptide sequence of CD8a (GenBank number AAH25715.1, aa 1-21) was fused to a human PAP-specific single chain variable fragment (scFv) derived from S5D1. Second, the scFv fragment was fused to the human CD8a transmembrane domain (GenBank number NP_001759.3, aa 138-206), followed by the human 4-1BB costimulatory domain (GenBank number NP_001552.2, aa 214-255), human CD3ζ signaling moieties (GenBank number NP_000725, aa 52-163), and green fluorescence protein (GFP, GenBank number AAB02572.1, aa 1-239). The CD19-CAR was constructed with a humanized antibody (L2D8, humanized from FMC63).17

Lentivirus production: Lentiviral plasmids and packing plasmids were transfected into 293T cells (Thermo, A35347). Virus-containing supernatant were collected 48 h after transfection and then concentrated and purified with a tangential flow filter (Repligen, S02-S05U-05-S). Lentivirus were stored at −80 °C, and their titers were determined using 293T cells.

Human T cell and B cell sorting: Peripheral blood mononuclear cells (PBMCs) were isolated from the blood of four healthy donors by gradient centrifugation in Lymphocyte Separation Medium (LSM, MP Biomedicals LLC, 50494). After washing twice with MACS buffer containing 5% human serum albumin (HSA, Miltenyi, 130-045-101/130-045-201), T cells were selected by using a CD4/CD8 separation kit (Miltenyi, 130-045-101/130-045-201) and suspended at 1 million cells/mL in TexMacs serum-free medium (Miltenyi, 170-076-309) supplemented with 12.5 ng/mL interleukin-2 (IL-2, R&D, 202-IL-500). B cells were selected by using a B Cell Isolation Kit II (Miltenyi, 130-091-151) and similarly cultured.

Generation of CAR-T cells: Twenty-four hours after separation and stimulation with Transact (Miltenyi, 130-111-160), T cells were transduced with lentiviruses encoding CD19 CAR or PAP CAR (with/without GFP). After 24 h of transduction, the supernatants were removed, and the culture media were changed every 48 h. Cells were maintained in TexMacs medium with 12.5 ng/mL IL-2.

PAP CAR-T cells in vivo assay: Six-week-old female NOG mice (Beijing Vital River) were subcutaneously injected with 2.0×106 PC3-tmPAP cells. Twenty days later (Day 0), the total 10 inoculated mice were randomly divided into a Non-transduced (NT, n=5) and PAP CAR-T (n=5) groups, receiving 1.0×106 NT or PAP CAR-T cells via tail vein injection. The individual mouse was considered the experimental unit within the studies. Retro-orbital blood was collected to assess the expansion of T cells in the peripheral blood by flow cytometry on Day 14. Tumor volumes were measured every 2-3 days until Day 21.

Cytotoxicity assay using the Agilent RTCA system: NT cell, CD19 CAR-T cells and PAP CAR-T cells were cultured in TexMacs medium supplemented with 12.5 ng/mL IL-2 until Day 9. To mimic the in vivo environment, on Day 9, cells were divided into groups and cultured alone or mixed with B cells in different ratios in TexMacs medium without IL-2. Meanwhile, PC3-tmPAP cells (1.0×104 per well) were cultured in a 96-well plate (xCELLigence RTCA system). After 48 hours, the previously cultured cells, either alone or in combination with B cells, were cocultured with the PC3-tmPAP cells according to their respective groups in the 96-well plate. The cell index was automatically measured every 15 mins for 120 h. The ratio of the difference between the values of the sample and control to the value of the control was defined as the specific lysis value.

Flow cytometry and immunohistochemistry: CAR expression on CAR-T cells was assessed using rabbit anti-human IgG (Jackson ImmunoResearch, 309-065-006) or mouse IgG (Jackson ImmunoResearch, 315-065-006). The monoclonal antibodies (mAbs) used for human T cells and PBMC staining included: CD45-PE (BD, 555483, clone: HI30), CD3-PerCPCy5.5 (Biolegend, 300430, clone: UCHT1), CD4-FITC (Biolegend, 317408, clone: OKT4), CD8-APC-CY7 (Biolegend, 344714, clone: SK1), CD19-APC (BD, 555415, clone: HIB19), CD45RA-A700 (BD, 560673, clone: HI100), CD62L-PE-CY7 (Biolegend, 304822, clone: DREG-56), CD69-FITC (Biolegend, 310904, clone: FN50), CD80-BV786 (BD, 564159, clone: L307.4), CD86-BV605 (BD, 562999, clone: FUN-1), CD137-APC (BD, 550890, clone: 4B4-1), PD-1-APC (BD, 558694, clone: MIH4), TIM3-APC-CY7 (Biolegend, 345044, clone: F38-2E2), LAG-3-PC5.5 (Biolegend, 369312, clone: 11C3C65), and goat anti-human IgG-AF488 (Abcam, Ab96907). A rabbit mAb (D3Y5P, #12861, Cell Signaling) was used for immunohistochemistry (IHC) analysis of PAP expression. Formalin-fixed, paraffin-embedded specimens were purchased in the form of commercially available tissue microarrays (HOrgN090PT02, Shanghai Outdo Biotech Company). PAP antibody (BioIntron, B21482901) was used to measure PAP expression on the surfaces of PC3 cells. CD3 (CST, #85061), CD4 (CST, #48274), CD8 (CST, 85336S), and GFP (CST, 2956S) antibodies were used for IHC analysis of tumor tissue sections from animal experiments. And QuPath (0.5.1) was used for analysis.

Cytokine cytometric bead array (CBA): In vitro coculture supernatants and murine peripheral blood plasma were collected, and cytokine levels were analyzed by flow cytometry microspheres (Beckman Cytoflex). FCAP Array software was used to analyze the levels of the cytokines, including IL-2 (BD, 558270), interferon gamma (IFN-γ, BD, 560111), tumor necrosis factor alpha (TNF-α, BD, 558273), and granzyme-B (BD, 560304).

CoupledCAR in vivo assay: Six-week-old female NOG mice were subcutaneously injected with 2.0×106 PC3-tmPAP cells. After 24 days (Day 0), the total 63 mice were randomly divided into 9 groups (n=7 per group): MOCK, NT, B cells, CD19 CAR-T, PAP CAR-T, PAP CAR-T+CD19 CAR-T, CD19 CAR-T+B cell, PAP CAR-T+B cell, and PAP CAR-T+CD19 CAR-T+B cell. Each group was infused via the tail vein with 200 L DPBS (Gibco, 14190-136), containing 1.0×106 NT cells, 1.0×106 B cells, 1.0×106 CD19 CAR-T cells, 3.3×105 PAP CAR-T cells, or their combinations as specified. On Day 3, 6 and 9, the mice in the B cell group, CD19 CAR-T+B cell group, PAP CAR-T+B cell group and PAP CAR-T+CD19 CAR-T+B cell group were infused with 1.0×106 B cells. The individual mouse was considered the experimental unit within the studies. Retro-orbital blood was collected on Day 14 to assess T cell expansion and cytokine levels by flow cytometry. Tumor volumes were measured every 3 days, and infiltration of CD3+, CD4+, CD8+, and PAP-CAR-T cells into tumors was assessed by immunohistochemistry.

Single-cell sequencing analysis: Single-cell sequencing was performed by NovelBio Bio-Pharm Technology Co. Ltd. The scRNA-Seq libraries and V(D)J libraries were generated using the 10× Genomics Chromium Controller Instrument and Chromium Single Cell 5′ library & gel bead kit, along with the V(D)J enrichment kit (10× Genomics, Pleasanton, CA). The cells (~1000 cells/μL) were loaded into each channel to generate single-cell gel bead-in-emulsions (GEMs). After reverse transcription, GEMs were disrupted, and barcoded cDNA was purified, amplified, and used to construct 5′ gene expression and TCR-enriched libraries. For 5′ library and the V (D) J library construction, the amplified barcoded cDNA was fragmented, A-tailed, ligated with adaptors and index PCR amplified. The final libraries were quantified using the Qubit High Sensitivity DNA assay (Thermo Fisher Scientific), and the size distribution of the libraries was determined using a High Sensitivity DNA chip on a Bioanalyzer 2200 (Agilent). All libraries were sequenced on an Illumina sequencer (Illumina, San Diego, CA) using a 150-bp paired-end run.

Barcode processing, alignment, and gene quantification were conducted using Cell Ranger Suite with GRCh38 as the reference genome. The pretreated mRNA data matrices were then imported into R, and downstream analysis was performed with the Seurat package. Low-quality cells (<200 genes, >2500 genes, or >10% mitochondrial genes) were filtered. Data were normalized and scaled using the Seurat package. T cells were classified as PAP CAR-T or CD19 CAR-T based on integrated gene fragments. Differential gene expression analysis was performed using nonparametric T-tests, and significant genes were visualized with ggplot2.

GSEA was conducted with the MAST R package using Kyoto Encyclopedia of Genes and Genomes (KEGG) gene sets from MSigDB database and T cell-related pathways. The CellChat R toolkit (Version 1.1.3) was used to assign cellular ligand-receptor interaction cycle scores to PAP CAR-T cells, CD19 CAR-T cells and B cells.

CellTrace expansion assay: Different combinations of CAR-T cells, NT cells and B cells were stained with CellTrace™ Violet (Thermo Fisher, C34557) or the CellTrace™ Far Red Cell (Thermo Fisher, C34564). Cell expansion was analyzed via flow cytometry every 24 hours for 144 hours.

Statistical analysis: GraphPad PRISM software was used for all the statistical analyses. The data are presented as the mean±SEM. Data shows technical replicates unless otherwise stated. Statistics were calculated using unpaired t-test, one-way ANOVA or two-way ANOVA as stated in the figure legends. Correlations were estimated by the calculation of 2-tailed Pearson coefficients and significance. All statistical analyses were performed with Prism software version 8.3.0 (GraphPad). The following symbols indicate statistical significance: P value<0.05=*, <0.01=**, <0.001=***, <0.0001=****; and P≥0.05=no significance=NS.

### Identification of PAP as a Potential Prostate Cancer Target for CAR-T Cell Therapy

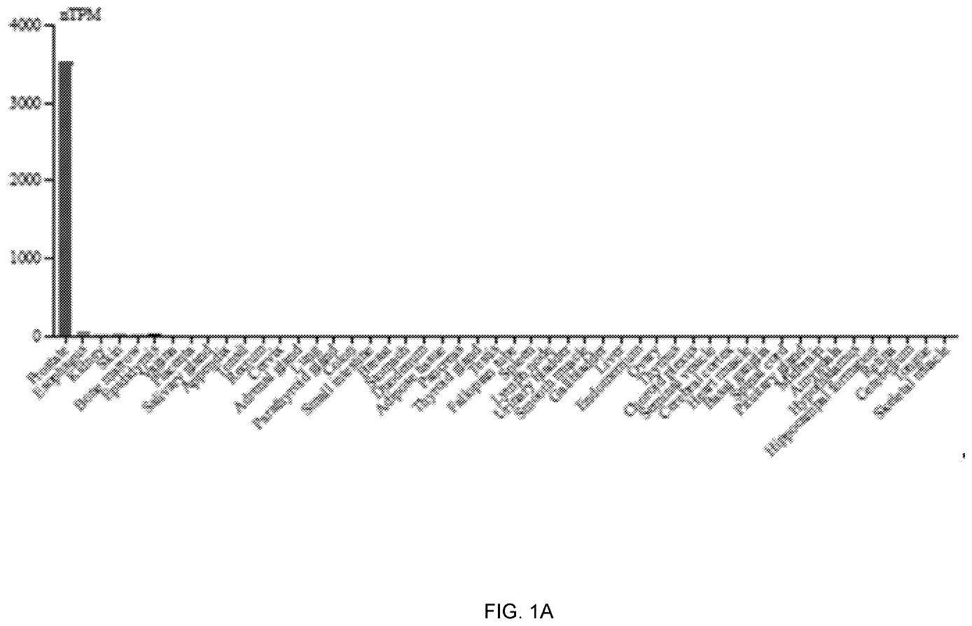

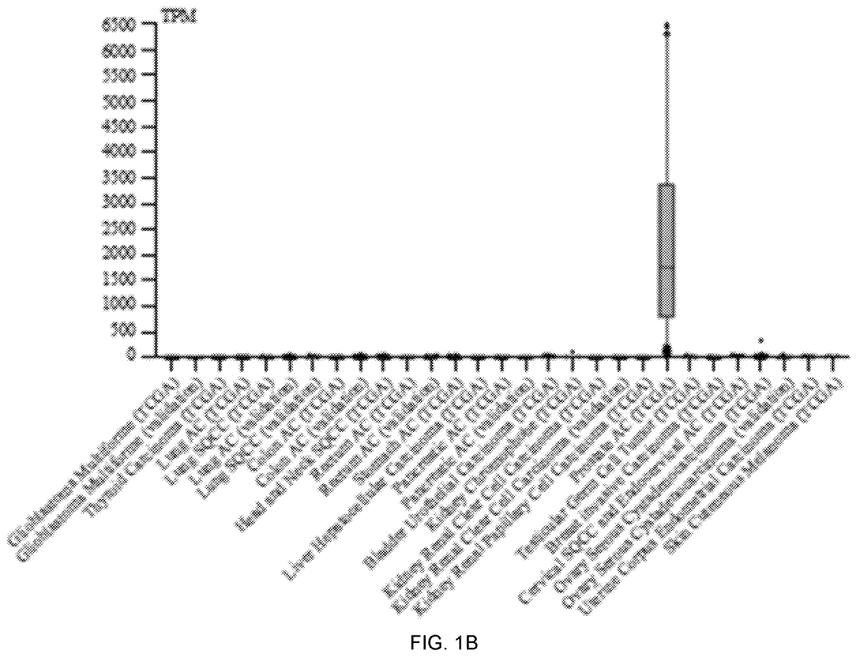

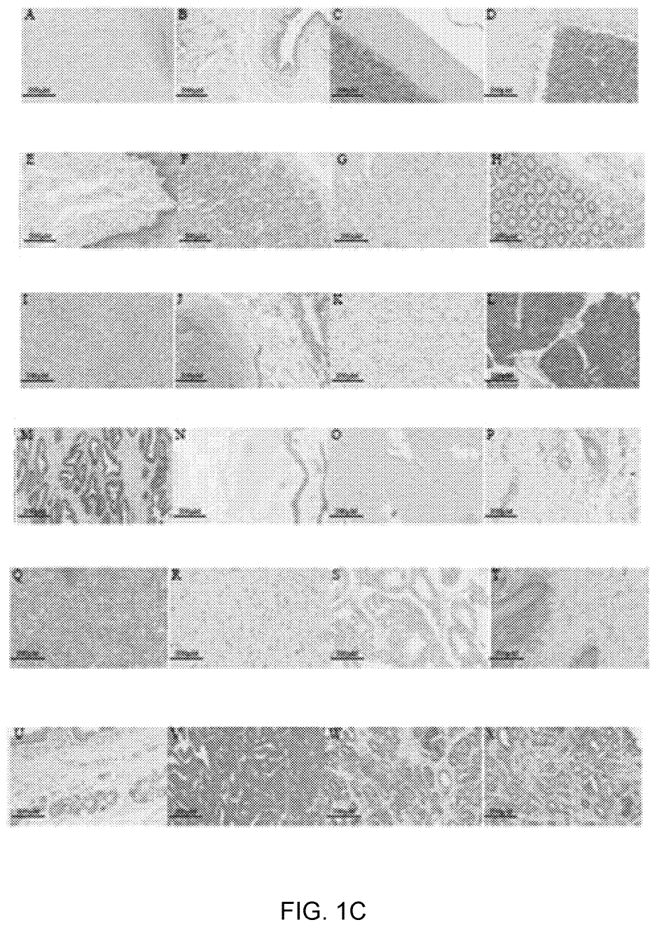

Based on the premise that the prostate is generally surgically removed from most patients with advanced prostate cancer, we tried to identify genes specifically expressed in both normal prostate cells and prostate tumor cells as targets for CAR-T cell therapy. Transcriptome data from the Human Protein Atlas database (www.proteinatlas.org/about/download) of all the genes that encode proteins expressed in 31 normal tissues and organs were downloaded, and the average protein-coding transcripts per million (pTPM) values of the genes in the tissue samples were collected to normalize the mRNA levels. A group of transmembrane genes specifically expressed in the prostate were defined and selected based on their mRNA levels being at least 5-fold higher than those in all other tissues. Among the selected genes, we found that PAP was specifically expressed in normal prostatic epithelial cells and prostate cancer cells but was expressed at very low or undetectable levels in other normal tissues (FIG. 1A).18 19 20 PAP expression was confirmed in 480 prostate cancer tissues based on an analysis of prostate cancer tissues from The Cancer Genome Atlas (TCGA) database (FIG. 1). PAP has two variants, tmPAP and secretory PAP.21 22 Here, we focused on tmPAP, which can be targeted by CAR-T cells. We verified that the PAP protein was specifically expressed on the membranes of normal prostate cells and prostate cancer cells but not those of other normal tissues by immunohistochemistry (IHC) staining (FIG. 1C). These results indicated that PAP is a specific prostate cancer target for CAR-T cell therapy.

### PAP-Specific CAR-T Cells Show Antitumor Activity Both In Vitro and In Vivo

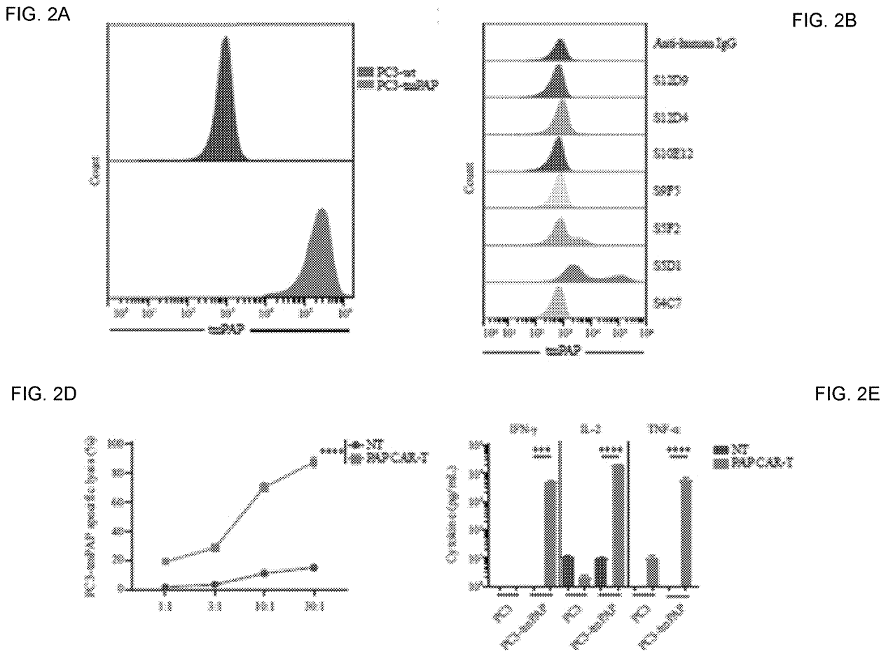

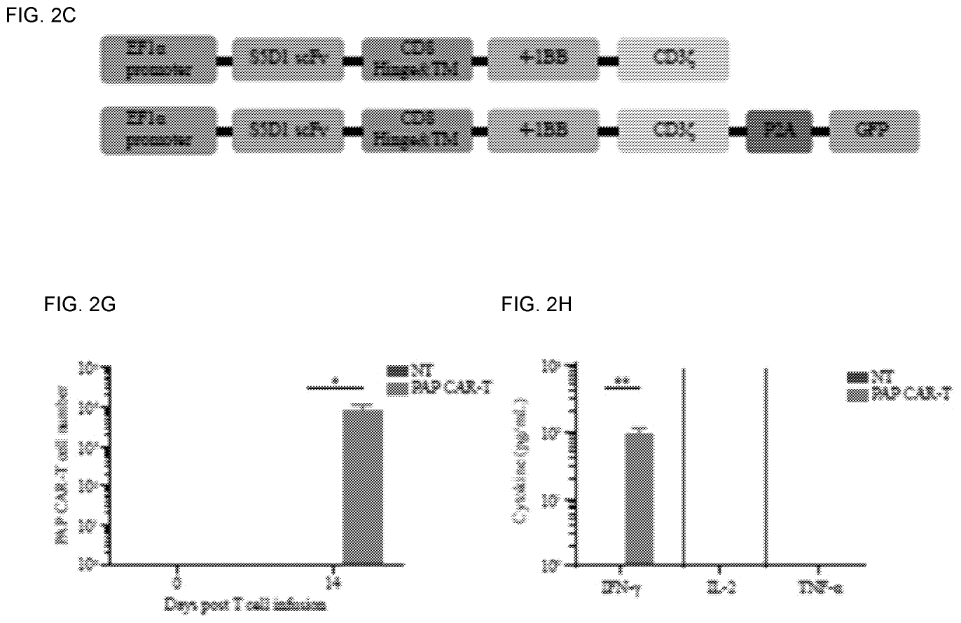

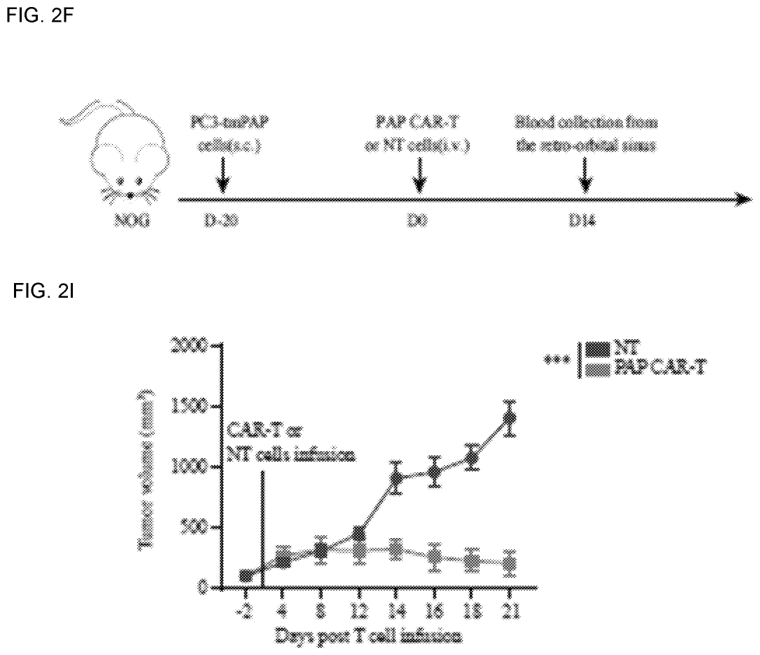

The PC3 cell line is commonly used to study prostate cancer.20 Some previous studies indicated that the PC3 cell line expressed PAP, while the majority of studies found that it does not.23 24 25 We verified that PAP is not expressed in the PC3 cell line. Therefore, we transduced the PC3 cell line with a lentiviral vector to express tmPAP and established the PC3-tmPAP cell line for PAP-specific antibody screening and the CAR-T cell activity study (FIG. 2A). To generate antibodies against PAP, we screened the human single-chain antibody library using recombinant tmPAP protein. After three rounds of screening, we identified seven single-chain antibodies that specifically bind to recombinant tmPAP-His antigen (FIG. 8A-B). We cloned the seven single-chain antibodies into eukaryotic expression vectors to generate fully humanized antibodies and all the antibodies demonstrated binding ability to the tmPAP-His antigen. Furthermore, we evaluated their binding ability to the PC3-tmPAP cell line. Interestingly, only two antibodies demonstrated binding to tmPAP, with S5D1 showing the best binding ability (FIG. 2B). The affinity measurement of the S5D1 antibody also showed high affinity (supplemental Fig. S1D). Based on scFv of S5D1, second-generation PAP-targeting CAR-T cells were constructed with or without the GFP reporter (FIG. 2C). We found that PAP CAR-T cells could efficiently kill PAP-positive cells (FIG. 2D) and release significant amounts of IFN-γ, IL-2, and TNF-α in vitro (FIG. 2E). No killing or detectable cytokine release was observed when PAP CAR-T cells were cocultured with PAP-negative cells (FIG. 2E). To assess the in vivo antitumor activity of PAP CAR-T cells, NOG mice were subcutaneously injected 2.0×106 PC3-tmPAP cells 20 days before the intravenous injection of 1.0×106 PAP CAR-T or NT cells (FIG. 2F). Significant expansion of PAP CAR-T cells in peripheral blood was observed on Day 14 (FIG. 2G). IFN-γ production was detected on Day 14 in the PAP CAR-T cell group (FIG. 2H). However, IL-2 and TNF-α were not detected in the peripheral blood of any group, may because these two cytokines were locally released. (FIG. 2H). The tumor volumes injected with PAP CAR-T cells were substantially reduced compared to those in the mice injected with NT cells (FIG. 2I). These results demonstrated that PAP is a valid target for CAR-T cells and that PAP CAR-T cells are very efficient in killing PAP-positive cells both in vitro and in vivo.

### Anti-CD19 CAR-T Cells Induce Remarkable Expansion of Non-Transduced Normal T Cells

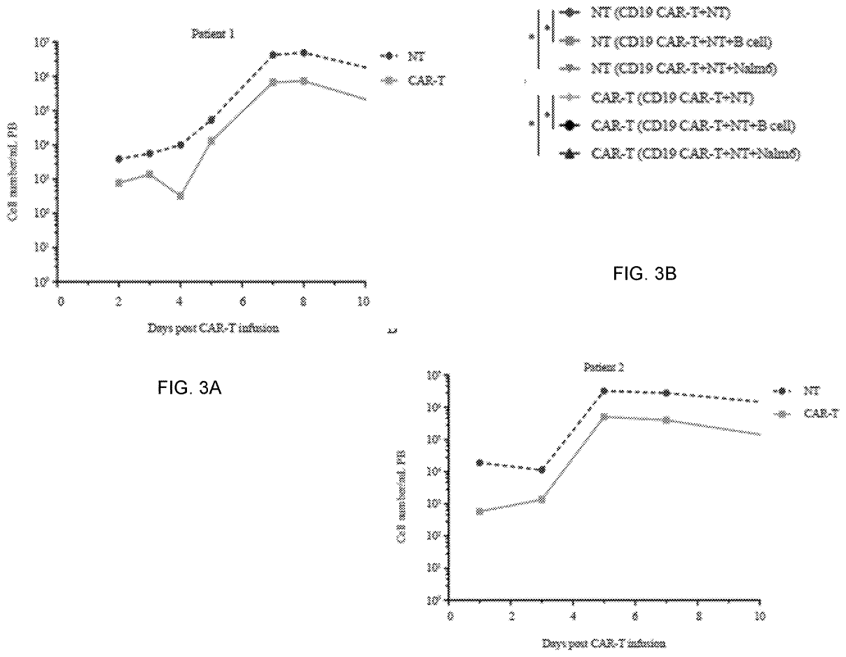

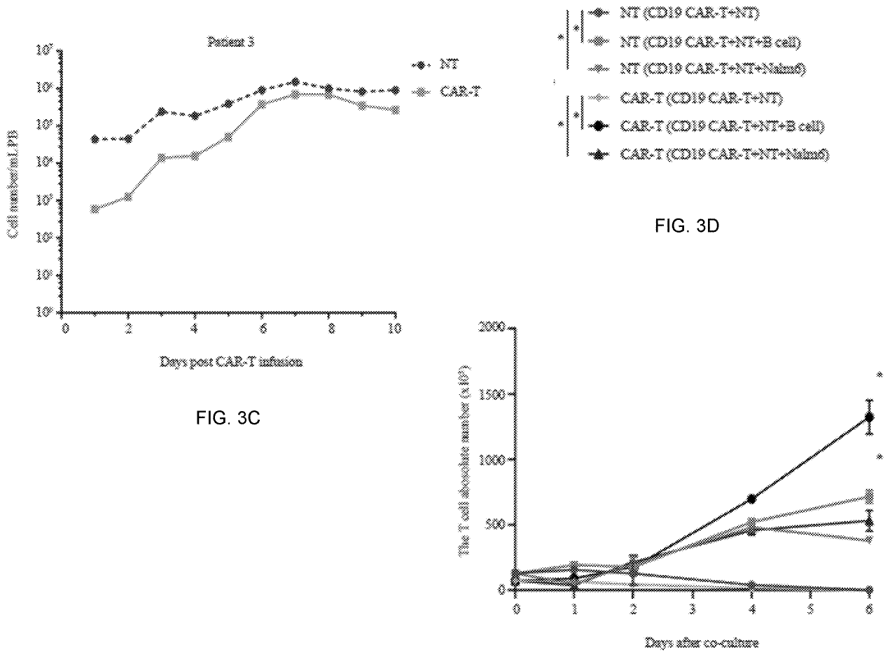

Although second-generation PAP CAR-T cells were shown to have a reasonable killing ability in the animal experiments, conventional solid tumor-targeting CAR-T cell therapies have shown limited efficacy in patients based on clinical trials.26 27 The lack of efficacy in solid tumors is due to the limited number of antigens outside the tumor microenvironment that can stimulate chimeric antigen receptor signaling, resulting in very poor CAR-T cell activation and expansion. Therefore, further improvements to the activation and/or expansion of solid tumor-targeting CAR-T cells without antigens may be helpful. In our previous clinical trial on the use of CD19 CAR-T cells in the treatment of leukemia,28 29 we unexpectedly observed a phenomenon in which NT cells were expanded together with CD19 CAR-T cells (FIGS. 3A-3C). In clinical trials of CD19 CAR-T for hematologic malignancies, we observed that CD19 CAR-T cells killing CD19-positive tumor cells or B cells can promote the expansion of NT cells in the absence of specific antigens for NT cells. To confirm whether this observed phenomenon is true, we established an in vitro verification experiment. We co-cultured CD19 CAR-T cells and NT cells together with or without B cells or CD19-positive tumor cells. We then measured the expansion of CD19 CAR-T and NT cells. The results showed that when CD19 CAR-T+NT cells were co-cultured with B cells or CD19-positive tumor cells, CD19 CAR-T cells expanded significantly, and as observed in the clinical trial, NT cells also expanded significantly without specific antigen stimulation. However, when NT and CD19 CAR-T cells were cultured alone, no expansion occurred (FIG. 3D). This is consistent with what was observed in the clinical trials of CD19 CAR-T. Therefore, we speculate that replacing NT cells with solid tumor CAR-T cells and co-culturing them with CD19-positive target cells would yield the same result.

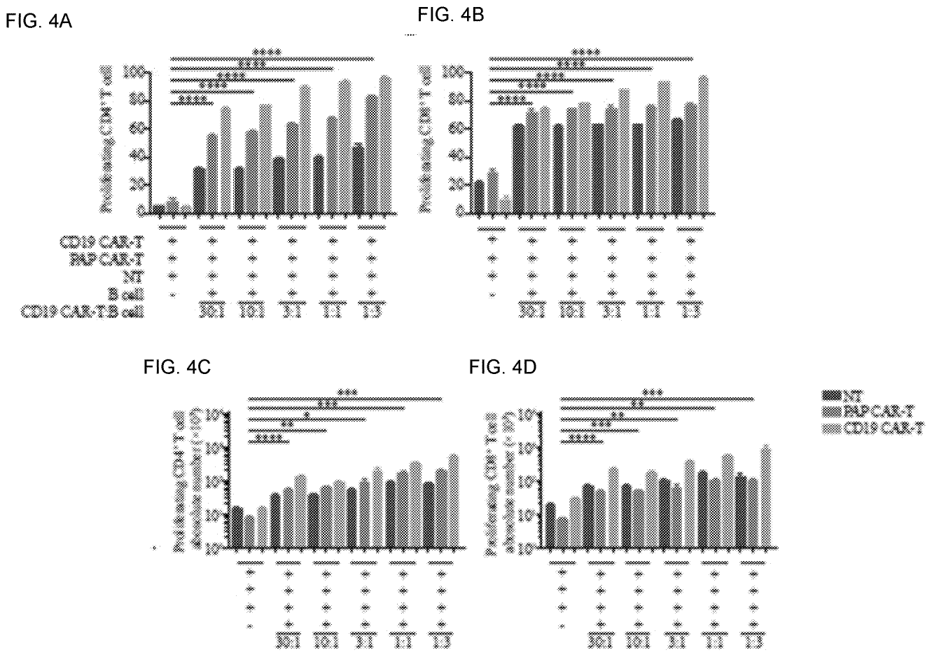

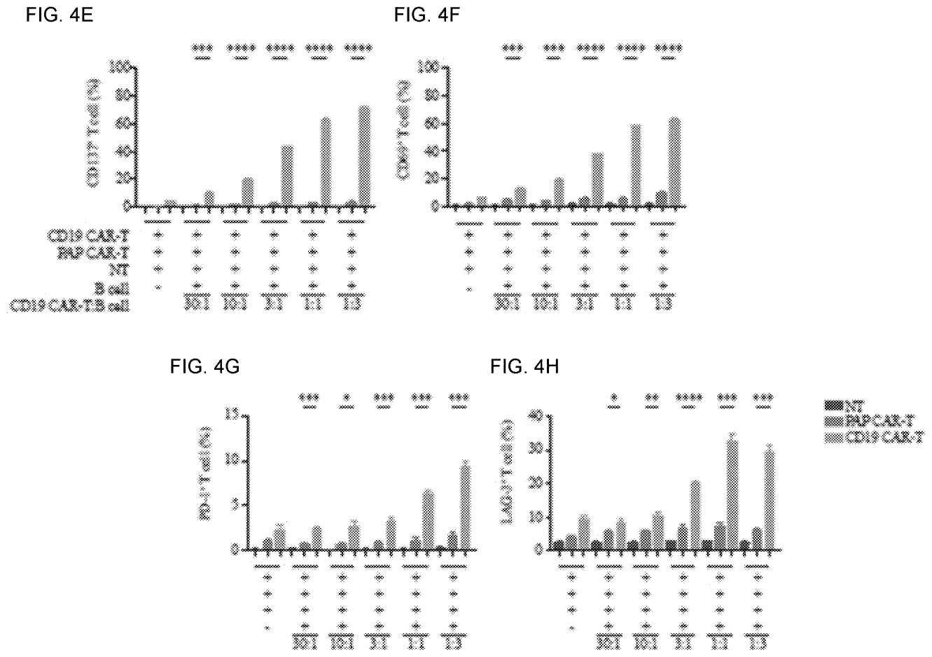

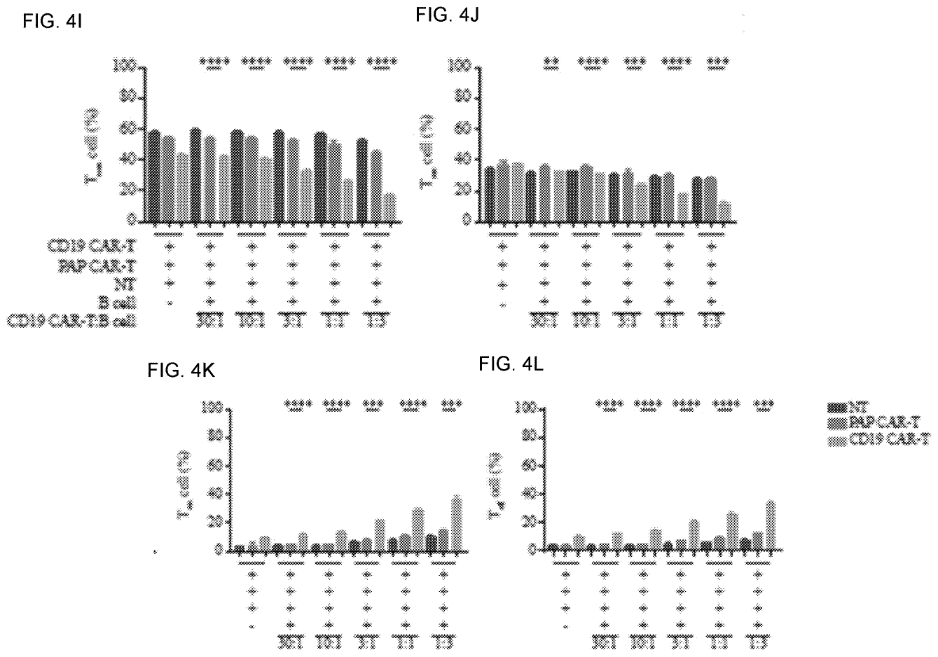

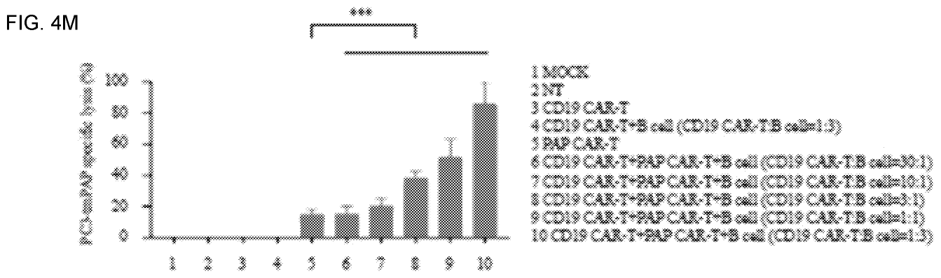

The CoupledCAR approach enhanced the expansion and functionality of PAP CAR-T cells in vitro. We conducted a coculture assay using three different cell populations: CD19 CAR-T cells, PAP CAR-T cells, and NT cells. Upon adding B cells, we observed a significant expansion in the populations of CD19 CAR-T cells, PAP CAR-T cells, and NT cells, both in terms of the percentage of proliferating cells and the absolute cell numbers. (FIG. 4A-4D). The expression levels of activation markers, such as CD137 and CD69, were shown to be upregulated in CD19 CAR-T cells, but NT cells and PAP CAR-T cells showed very slight upregulation (FIG. 4E-4F). Moreover, CD19 CAR-T cells exhibited significantly upregulated levels of the exhaustion markers PD-1 and LAG-3, while PAP CAR-T and NT cells had much lower expression levels of these exhaustion markers (FIGS. 4G and 4H). In addition, PAP CAR-T cells and NT cells maintained higher percentages of cells with the stem-like central memory T cell (CD45ROnegCD62Lpos) and central memory T cell (CD45ROposCD62Lpos) phenotypes and lower percentages of cells with the effector memory cell (CD45ROnegCD62Lneg) and effector T cell (CD45ROposCD62Lneg) phenotypes than CD19 CAR-T cells (FIG. 4I-4L), while CD19 CAR-T cells exhibited a higher percentages of cells with the effector T cell (CD45ROposCD62Lneg) phenotype. In the cytotoxicity assay, the PAP CAR-T cells+CD19 CAR-T cells+B cells groups with different CD19 CAR-T: B cell ratios showed better tumor cell lysis abilities than the other groups (FIG. 4M). These data suggest that PAP CAR-T cells could expand without antigen, exhibit lower exhaustion and higher memory-like status, and achieve better killing when cocultured with CD19 CAR-T cells and B cells. We named this system for expanding solid tumor-targeting CAR-T cells CoupledCAR, and this system includes CD19 CAR-T cells and solid tumor-targeting CAR-T cells.

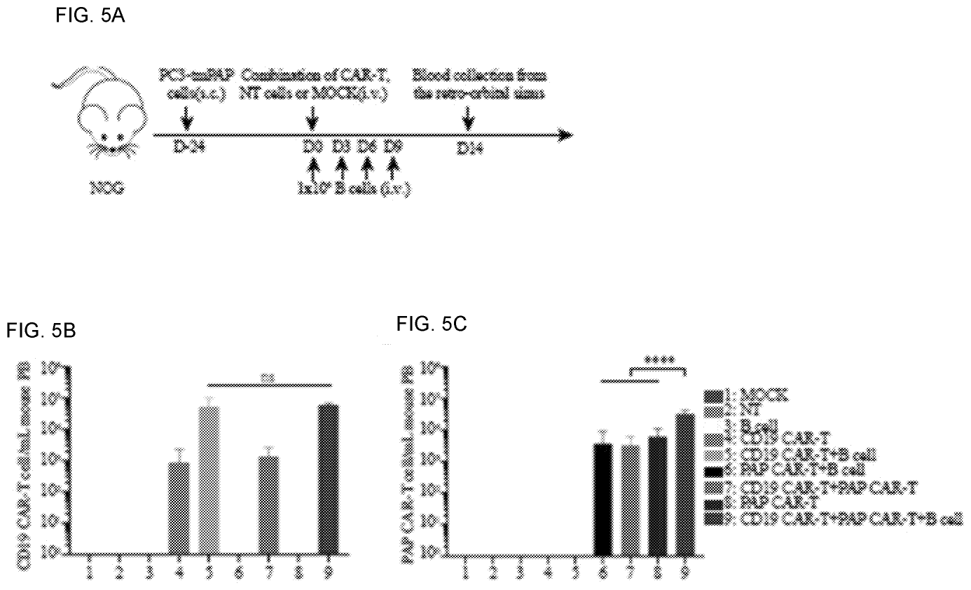

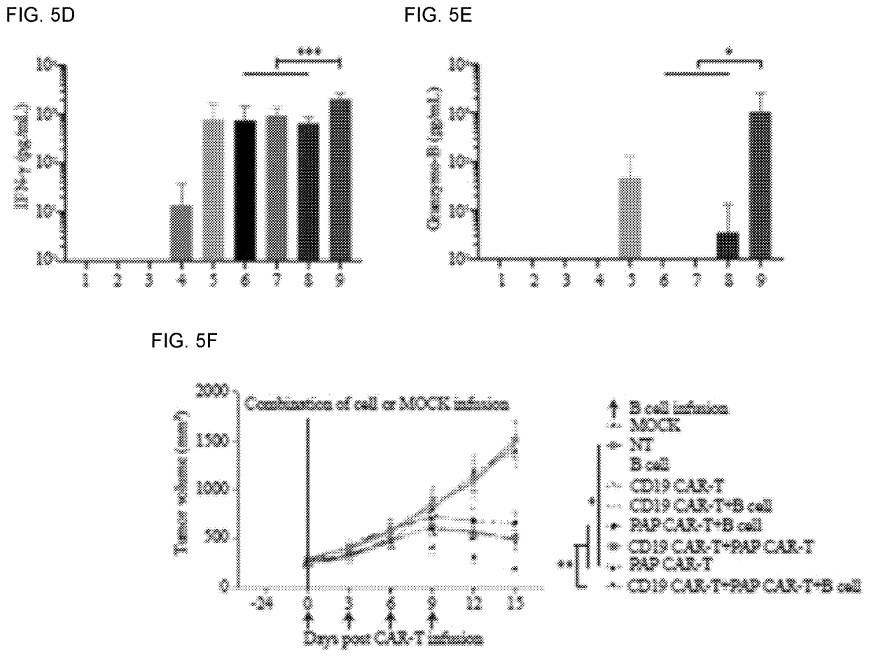

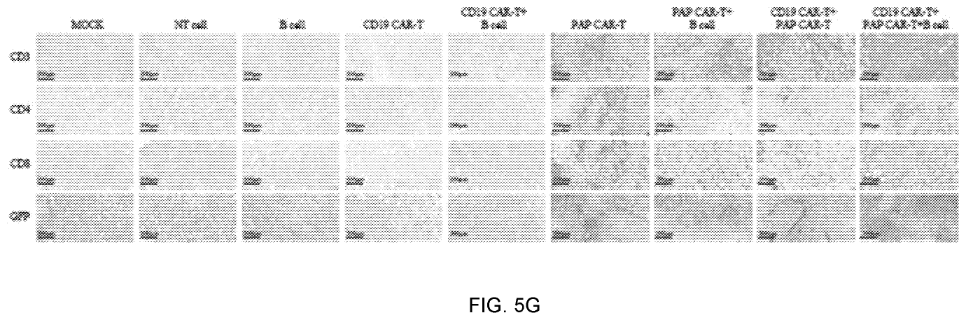

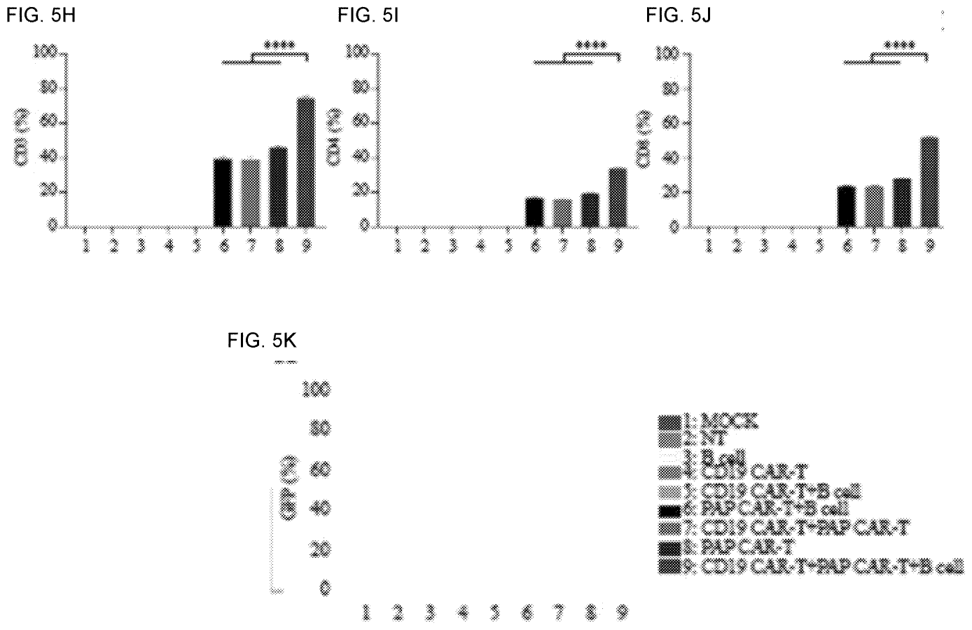

The CoupledCAR approach enhanced the efficient expansion and enhanced the functionality of PAP CAR-T cells in vivo. To determine whether the CoupledCAR approach enhances the anti-solid tumor efficacy of PAP CAR-T cells in vivo, we tested the CoupledCAR system in NOG mice with subcutaneous tumors. CD19 CAR-T cells or PAP CAR-T cells were infused alone or in combination via the tail vein 24 days after the transplantation of tumor cells, and selected groups were infused with B cells at 4 time points (FIG. 5A). CD19 CAR-T cells were found to expand rapidly upon encountering B cells in both the CD19 CAR-T cell+ B cell group and the CD19 CAR-T cell+ PAP CAR-T cell+ B cell group (FIG. 5B). The PAP CAR-T cells in peripheral blood in the CD19 CAR-T cell+ PAP CAR-T cell+ B cell group expanded by more than 5-fold compared to those in the PAP CAR-T cell group on Day 14 (FIG. 5C). The peripheral blood levels of IFN-γ and granzyme B were significantly increased in the CD19 CAR-T cell+ PAP CAR-T cell+ B cell group (FIG. 5D-5E). Moreover, the CD19 CAR-T cell+ PAP CAR-T cell+ B cell group showed better tumor killing than all the other groups. (FIG. 5F). In addition, the CD19 CAR-T cell+ PAP CAR-T cell+ B cell group showed the highest degree of CD3+, CD4+, CD8+, and PAP CAR-T cell infiltration into tumors (FIG. 5G-5K). In conclusion, the CD19 CAR-T cell+PAP CAR-T cell+B cell group showed better CAR-T cell expansion, infiltration and antitumor effects than all the other groups.

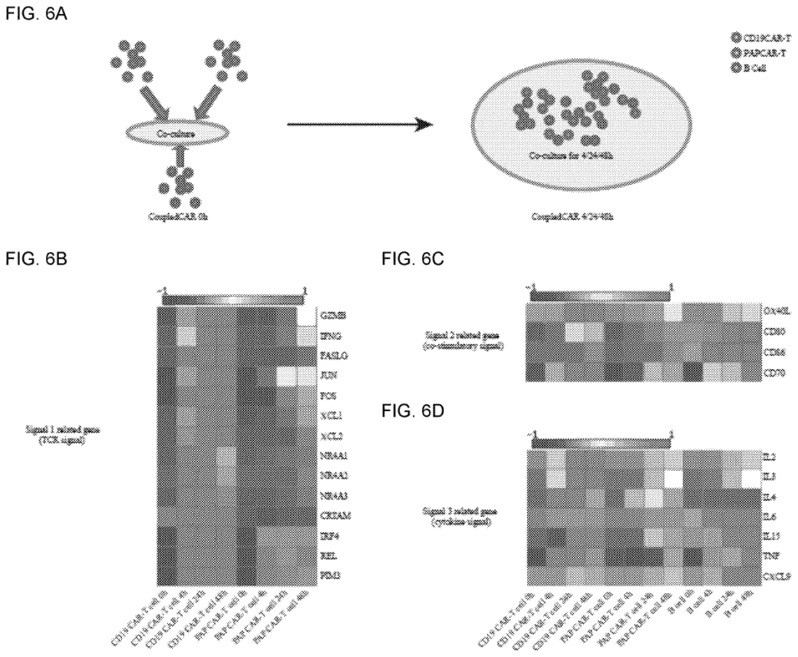

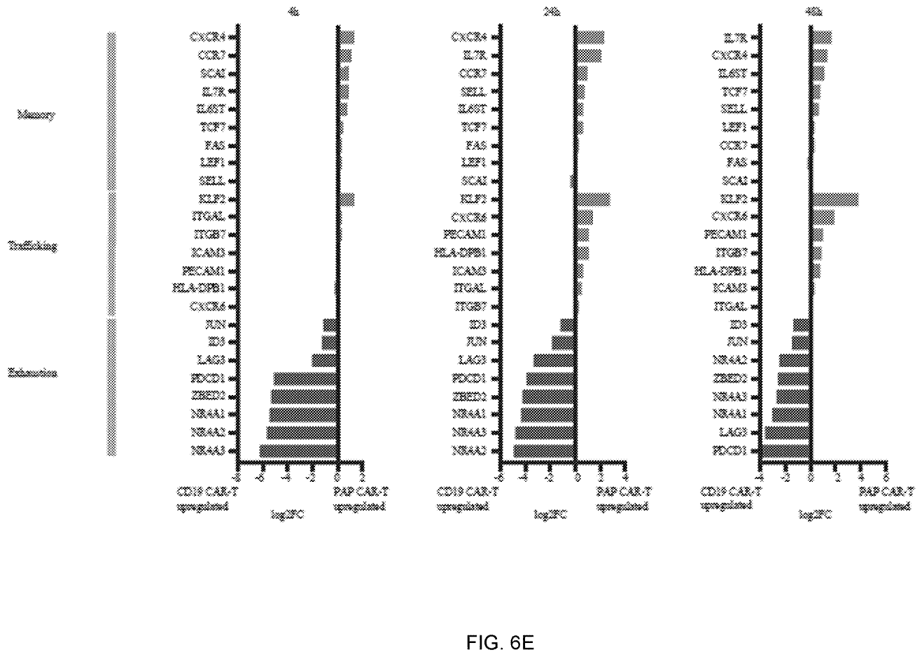

Expansion of PAP CAR-T cells was mediated by costimulatory and cytokine/chemokine signaling. To determine the mechanism by which PAP CAR-T cells substantially expand in the CoupledCAR system, we conducted single-cell RNA sequencing analysis. We sequenced the mRNA of each single cell from a coculture assay at different time points (FIG. 6A).

Previous studies showed that the expansion and activation of T cells depended on three signals, including T-cell receptor (TCR) signals (Signal 1), costimulatory signals (Signal 2) and cytokine signals (Signal 3).30-33 We found that TCR signaling and costimulatory signaling were highly activated in CD19 CAR-T cells at 4, 24, and 48 h (FIGS. 6B and C). Interestingly, PAP CAR-T cells exhibited no TCR signaling activation (FIG. 6B). The expression of the CD80 and CD86, ligands for CD28, were significantly upregulated in B cells at 24 and 48 h, and the expression of the CD70, ligand for CD27, was significantly upregulated in both B cells and CD19 CAR-T cells at 24 and 48 h, and also, the expression of the OX40L, ligands for OX40, was significantly upregulated in CD19 CAR-T cells at 24 and 48h (FIG. 6C), providing a source of the costimulatory signal.33 Moreover, the expression levels of cytokines or chemokines that promote T cell expansion, such as IL-2, IL-3, IL-4, IL-6, IL-15, TNF and CXCL9, 34-37 were significantly upregulated in CD19 CAR-T cells or B cells (FIG. 6D). Our data suggest that PAP CAR-T cell expansion should be mediated by Signal 2 and Signal 3, which are provided by CD19 CAR-T cells and B cells, but not by Signal 1.

Previous studies have shown that TCR-based expansion results in CAR-T cell exhaustion, while an expansion approach based on Signals 2 and 3 results in reduced exhaustion.38 39 The expression of genes associated with exhaustion and memory were analyzed, and we found that PAP CAR-T cells showed a memory-like status and reduced exhaustion (FIG. 6E), which can enhance the efficacy of CAR-T cells.39 40 The expression of the memory-related genes CCR7, IL7R, SELL, TCF7 and LEF1 etc. were upregulated in PAP CAR-T cells and the expression of the exhaustion-related genes PDCD1, LAG-3, ID3, NR4A1, NR4A2, and NR4A3 etc. were downregulated in PAP CAR-T cells compared with CD19 CAR-T cells.39 Unexpectedly, we found that the expression of the trafficking-related genes KLF2, CXCR6, ITGAL, and ITGB7 etc. were also significantly upregulated (FIG. 6E), suggesting that PAP CAR-T cells had better infiltration ability than CD19 CAR-T cells.41 42 This is consistent with the results mentioned above (FIG. 5G). These data suggest that PAP CAR-T cells exhibit lower exhaustion and maintain higher memory-like status in CoupledCAR system, which may result in a better anti-tumor ability.

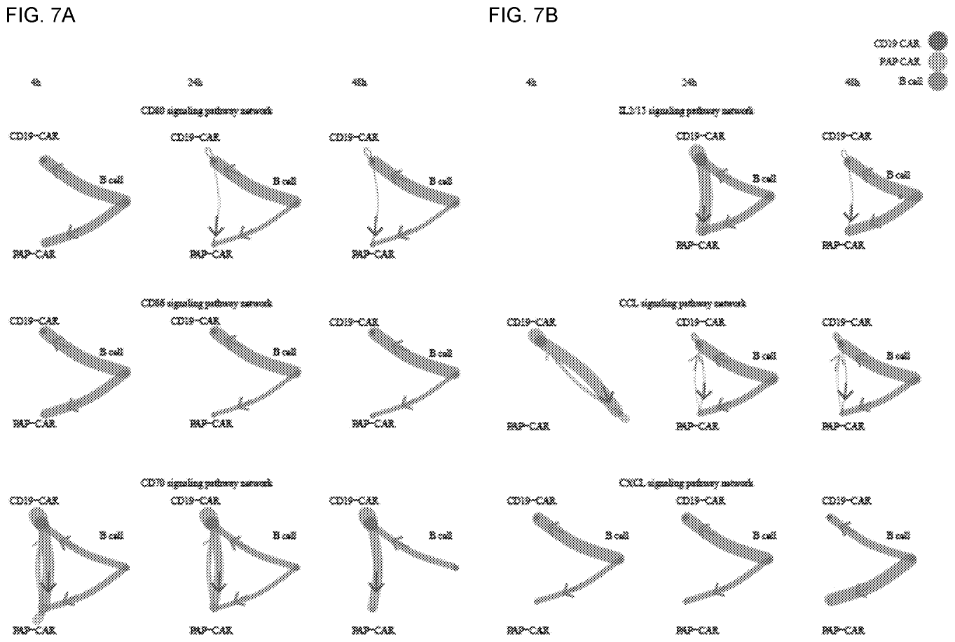

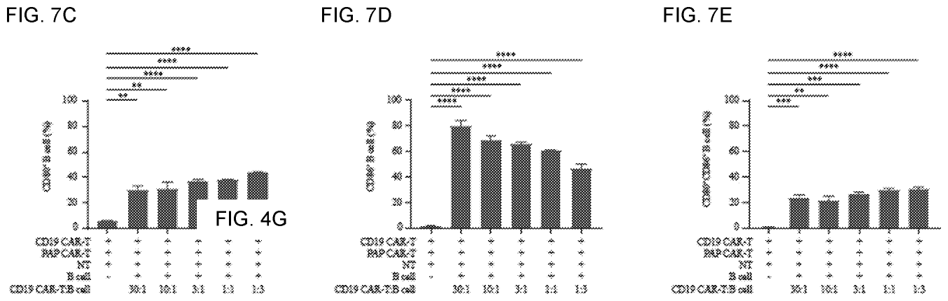

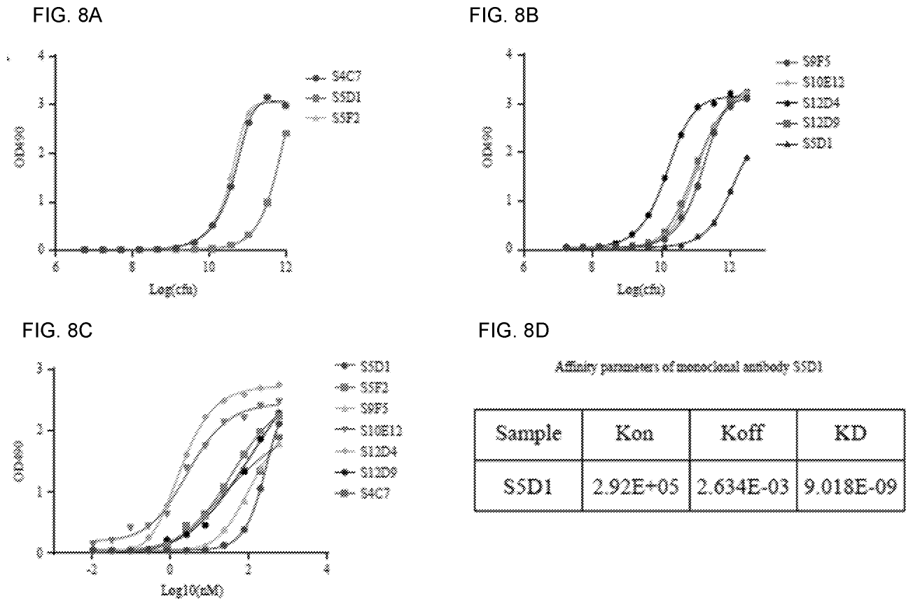

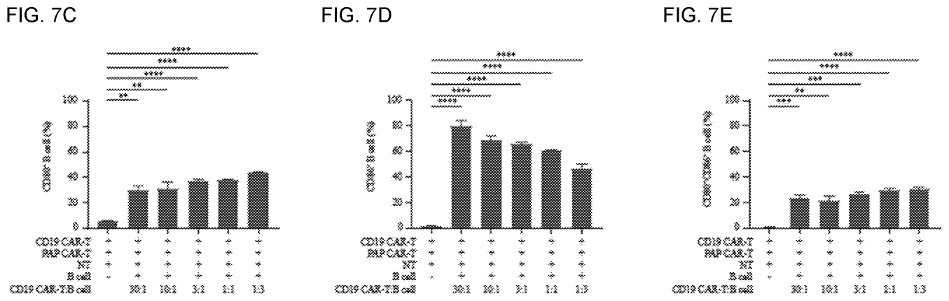

Analysis of cell-cell communication using CellChat. To verify the signaling networks, the R toolkit CellChat was used to analyze ligand-to-receptor interactions among PAP CAR-T cells, CD19 CAR-T cells and B cells.43 The results showed that PAP CAR-T cells received CD80 and CD86 signals mainly from B cells at 4 h, 24 h and 48 h and received decreased CD80 signals from CD19 CAR-T cells at 24 h and 48 h (FIG. 7A). PAP CAR-T cells also received CD70 signals from B cells at 4 h and 24 h and received CD70 signals from CD19 CAR-T cells at 4 h, 24 h and 48 h (FIG. 7A). We found that both B cells and CD19 CAR-T cells delivered IL-2/15-mediated signals to PAP CAR-T cells at 24 h and 48 h (FIG. 7B). Moreover, B cells delivered CXCL-mediated signals starting at 4 h (FIG. 7B). These data indicate that CD19 CAR-T cells and B cells provide Signals 2 and 3 to PAP CAR-T cells. To further demonstrate that B cells indeed provide the second signal in the CoupledCAR system, we established an in vitro co-culture experiment involving PAP CAR-T cells, CD19 CAR-T cells, and B cells. After 24 hours, we used flow cytometry to assess the expression of the co-stimulatory molecules CD80 and CD86 on the surface of B cells to evaluate whether B cells can provide the second signal. The results showed that after 24 hours of co-culture, the expression of CD80 and CD86 on the surface of B cells was significantly upregulated. This indicates that B cells have been activated and may serve as antigen-presenting cells, indeed providing co-stimulatory signals to assist in the expansion of PAP CAR-T cells.

In conclusion, our study shows that PAP CAR-T cells were expanded in the CoupledCAR system by costimulatory signals and cytokine/chemokine signals without stimulation by antigens, and these cells showed less exhaustion, more memory-like status, and better trafficking abilities than conventional CAR-T cells.

Our findings have the following important significance. First, our study indicates that PAP is a specific and valid target for CAR-T therapy and may be a potentially better target than PSMA due to its stringent specificity. Although PAP is a biomarker for prostate cancer and the target of Provenge, a cancer vaccine by Dendreon,19 44 previous studies suggested it could not be used as a target for antibody drugs or cell therapies for prostate cancer, 21 45 because PAP was reported to be not specific to the prostate but rather also expressed in normal bladder, kidney, pancreas, cervix, testis, lung and ovary tissues, which limits its usefulness as a specific target for prostate cancer.45 46 47 However, these earlier findings were likely influenced by false-positive staining caused by the non-specificity of polyclonal antibodies used in those studies.48 In contrast, our database analysis and IHC staining based on specific antibody confirmed that PAP is expressed in only normal prostate epithelial cells and not in any other normal tissues. Our study indicates that PAP is a suitable prostate cancer target and can be used for CAR-T cell therapy, which may greatly increase the prospect of clinical application. Moreover, the strategy of screening surface antigens expressed on corresponding normal tissues that are dispensable for life, such as the prostate, may serve as a novel method for expanding possible solid tumor-specific targets.

Second, we showed that the CoupledCAR approach provides a novel and feasible strategy for expanding CAR-T cells without any direct antigen stimulation.13 14 Recently, different approaches have been utilized to promote the antigen-specific expansion of solid tumor-targeting CAR-T cells. While antigen-dependent expansion methods can be used to expand solid tumor-targeting CAR-T cells, these methods simultaneously may cause rapid cell exhaustion due to direct stimulation via CD3ζ signaling. CAR-T cell exhaustion leads to insufficient therapeutic efficacy, while memory-like status CAR-T cells are more capable of sustaining persistence and survival, which enhances their anti-tumor activity and efficacy.49 In contrast, the CoupledCAR approach enhances the memory-like status of CAR-T cells and does not lead to rapid cell exhaustion, which may enhance their efficacy in the treatment of solid tumors. Additionally, the adverse events induced by the CD19 CAR-T cell-mediated elimination of B cells were minimal, and elimination of regulatory B cells from the solid tumor microenvironment may be beneficial in the treatment of solid tumors.50 Therefore, the antigen-independent expansion of solid tumor-targeting CAR-T cells may be superior to antigen-dependent expansion regarding to the killing of solid tumor cells in patients.

Finally, the antigen-independent expansion approach is not based on a specific tumor antigen, suggesting that it should work as a platform to support not only PAP CAR-T therapies but also other solid tumor-targeting CAR-T therapies. The tumor microenvironment is a barrier that limits not only CAR-T cell therapy but also other types of adoptive T cell therapy that also require direct physical interactions between T cells and tumor-specific target antigen(s) for the activation and expansion of T cells. Therefore, this novel approach can also be incorporated into TCR-T or tumor-infiltrating lymphocyte (TIL)-based therapies to facilitate the expansion of infused T cells in vivo. Moreover, CD19 CAR-T cells in the CoupledCAR system may also be replaced with CAR-T cells that target other white blood cells antigens (e.g., CD20, CD22, and BCMA) as long as the CAR-T cells can engage these targets in the circulating blood. This strategy may provide feasible and flexible opportunities for solid tumor cell therapies.

This antigen-independent expansion approach is the key for advancing CAR-T cell therapies against solid tumors. CoupledCAR approach represents a universal platform technology that can be effectively extended to other solid tumor CAR-T applications. Here, we are currently conducting clinical studies to further validate the efficacy of PAP-targeted CoupledCAR-T therapy in prostate cancer patients.

List of Abbreviations: CAR-T: Chimeric antigen receptor T; PAP: Prostatic acid phosphatase; TCGA: The Cancer Genome Atlas; IHC: Immunohistochemistry; scRNA-Seq: single cell RNA sequencing; TCR: T-cell receptor; PSMA: Prostate-specific membrane antigen; BCMA: B cell maturation antigen; GCC: Guanylyl cyclase-c; mCRC: metastatic colorectal cancer; tmPAP: Transmembrane prostatic acid phosphatase; scFv: Single chain variable fragment; GFP: Green fluorescence protein; FBS: Fetal bovine serum; PBMCs: Peripheral blood mononuclear cells; LSM: Lymphocyte Separation Medium; HSA: Human serum albumin; NT: Non-transduced; IgG: Immunoglobulin G; mAbs: Monoclonal antibodies; CBA: Cytometric bead array; IFN-γ: Interferon gamma; IL: Interleukin; TNF-α: Tumor necrosis factor alpha; DPBS: Dulbecco's phosphate-buffered saline; GEMs: Gel bead-in-emulsions; PCR: Polymerase chain reaction; KEGG: Kyoto Encyclopedia of Genes and Genomes; pTPM: Protein-coding transcripts per million; TIL: Tumor-infiltrating lymphocyte.

FIGS. 1A-1C. Identification of PAP as a potential prostate cancer target for CAR-T cell therapy. (FIG. 1B A) Expression levels of PAP transcripts in various normal tissues from the Human Protein Atlas database are shown from highest to lowest. (FIG. 1B) Expression levels of transcripts in different cancer tissues from the Human Protein Atlas database. (FIG. 1C) IHC analysis of PAP protein expression in normal human tissues: A, artery; B, bladder; C, brainstem; D, cerebellum; E, esophagus; F, gastric mucosa; G, heart muscle; H, intestine; I, liver; J, lung; K, medulla oblongata; L, pancreas; M, prostate; N, seminal vesicle; O, skeletal muscle; P, skin; Q, spleen; R, telencephalon; S, testis; T, tongue; U, trachea; V-X, three human prostate cancer (PCa) samples. Scale bars represent 200 m.

FIGS. 2A-2I. PAP-specific CAR-T cells show antitumor activity both in vitro and in vivo. (FIG. 2A) The expression of tmPAP on PC3 and PC3-tmPAP cells. (FIG. 2B) The binding ability of antibodies to the tmPAP antigen on PC3-tmPAP cells. (FIG. 2C) Schematic diagram of the design of PAP CAR. (FIG. 2D) Killing ability of PAP CAR-T and None-transduced T (NT) cells. (FIG. 2E)The concentrations of IFN-γ, IL-2 and TNF-α in the cell coculture supernatants. (FIG. 2F) Schematic illustration of the in vivo studies. (FIG. 2G) The expansion of CAR-T cells in the peripheral blood of mice in both the PAP CAR-T and NT groups on Day 14. (FIG. 2H) IFN-γ release, IL-2 release and TNF-α release in the peripheral blood in both the PAP CAR-T and NT groups on Day 14. (FIG. 2I) Tumor volumes in the mice over time (An unpaired t-test was used to compare between NT and PAP CAR-T on Day 21). P value<0.05=*, <0.01=**, <0.001=***, <0.0001=****; P>0.05=no significance=NS.

FIGS. 3A-3D. Anti-CD19 CAR-T cells induce remarkable expansion of non-transduced normal T cells. (FIGS. 3A-3C) Data from previous multiple-center clinical trials on the use of CD19 CAR-T cells for the treatment of NHL revealed substantial expansion of both CD19 CAR-T cells and NT cells in patients after CD19 CAR-T cell infusion. (FIG. 3D) An in vitro co-culture model was established by culturing CD19CAR-T and NT cells either alone or co-cultured with B cells or CD19-positive NALM6 cells at an E:T ratio of 3:1 for 6 days. The expansion of CD19CAR-T and NT cells from DAY 0 to DAY 6 post-co-culture was assessed and analyzed, and the results were compared with those of the cells cultured alone. Fig D was analyzed using two-way ANOVA. P value<0.05=*, <0.01=**, <0.001=***, <0.0001=****; P>0.05=no significance=NS.

FIGS. 4A-4M. The CoupledCAR approach enhanced the expansion and functionality of PAP CAR-T cells in vitro. (A-D) After 96 h of coculture with different CD19 CAR-T cells and the B cell ratio, percentage of CD4+ T cell (FIG. 4A) and CD8+ T cell (FIG. 4B) expansion, and absolute cell numbers of expanded CD4+ T cells (FIG. 4C) and CD8+ T cells were determined by flow cytometry (FIG. 4D). (FIGS. 4E, 4F) The expression levels of CD137 (FIG. 4E) and CD69 (FIG. 4F) in PAP CAR-T cells, NT cells, and CD19 CAR-T cells. (FIGS. 4G-4H) The expression of PD-1 (FIG. 4G) and LAG-3 (FIG. 4H) in PAP CAR-T cells, NT cells, and CD19 CAR-T cells. (FIGS. 4I-4L) The percentages of stem-like central memory T cells (CD45ROnegCD62Lpos) (FIG. 4I), central memory T cells (CD45ROposCD62Lpos) (FIG. 4J), effector memory T cells (CD45ROnegCD62Lneg) (FIG. 4K) and effector T cells (CD45ROposCD62Lneg) (FIG. 4L). (FIG. 4M) NT cells, PAP CAR-T cells, and CD19 CAR-T cells were grouped and either cultured alone or pre-mixed with B cells in different ratios, followed by coculture with PC3-tmPAP cells. The specific lysis against PC3-tmPAP tumor cells was analyzed. FIGS. 4A-4L were analyzed using an unpaired t-test. FIG. 4M was analyzed using one-way ANOVA.P value<0.05=*, <0.01=**, <0.001=***, <0.0001=****; P>0.05=no significance=NS.

FIGS. 5A-5K. The CoupledCAR approach enhanced the efficient expansion and enhanced the functionality of PAP CAR-T cells in vivo. (FIG. 5A) Schematic illustration of the in vivo studies. The prostate cancer xenograft model was established by the subcutaneous injection of 2.0×106 PC3-tmPAP cells, followed by the administration of different T and B cell or MOCK infusion strategies 24 days later (DO). The mice were divided into the MOCK group (n=7), NT group (n=7), B cell group (n=7), PAP CAR-T group (n=7), PAP CAR-T cell+CD19 CAR-T cell group (n=7), CD19 CAR-T cell group (n=7), CD19 CAR-T cell+ B cell group (n=7), PAP CAR-T cell+ B cell group (n=7) and PAP CAR-T cell+CD19 CAR-T cell+ B cell group (n=7). A total of 1.0×106 B cells were infused on Day 3, 6 and 9 in the groups that received B cell infusion. (FIGS. 5B-5G) Peripheral blood was collected on Day 14 to analyze CAR-T cell expansion (FIGS. 5B, 5C) and cytokine release (FIGS. 5D, 5E). The tumor volumes were measured every 3 days. (An unpaired t-test was used to compare between PAP CAR-T cell+CD19 CAR-T cell+ B cell group and PAP CAR-T group, PAP CAR-T cell+CD19 CAR-T cell+ B cell group and PAP CAR-T cell+CD19 CAR-T cell group, PAP CAR-T cell+CD19 CAR-T cell+ B cell group and PAP CAR-T cell+ B cell group on Day 15) (FIG. 5F). (FIG. 5G) The infiltration of CD3+, CD4+, CD8+, and PAP-CAR-transduced T cells into mouse tumors was assessed by immunohistochemistry. Scale bars represent 200 m. (FIGS. 5H-5K). The histograms show the infiltration of CD3, CD4, CD8, and PAP CAR-T cells in tumor tissues. Fig B was analyzed using an unpaired t-test. Fig C-F and H-K were analyzed using one-way ANOVA.P value<0.05=*, <0.01=**, <0.001=***, <0.0001=****, P>0.05=no significance=NS.

FIGS. 6A-6E. Expansion of PAP CAR-T cells was mediated by costimulatory and cytokine/chemokine signaling. (FIG. 6A) Schematic of the experimental design for single-cell sequencing using 10× Genomics. (FIGS. 6B-6D) Heatmap of TCR signaling (B), costimulatory signaling (FIG. 6C) and cytokine/chemokine signaling (FIG. 6D) of CD19 CAR-T cells, PAP CAR-T cells and B cells in the CoupledCAR system at 0 h, 4 h, 24 h and 48 h. (FIG. 6E) Heatmap of the distinct phenotypes of CD19 CAR-T cells and PAP CAR-T cells after activation via different mechanisms in the CoupledCAR system.

FIGS. 7A-7E. Analysis of cell-cell communication using CellChat. (FIG. 7A) CellChat analysis of cellular costimulatory interactions among CD19 CAR-T cells, PAP CAR-T cells and B cells at 4 h, 24 h and 48 h. (FIG. 7B) CellChat analysis of cellular cytokine/chemokine interactions among CD19 CAR-T cells, PAP CAR-T cells and B cells at 4 h, 24 h and 48 h. (FIGS. 7C-7E) After 24 h of coculture with different CD19 CAR-T cells and the B cell ratios in CoupledCAR system like in FIG. 4A-L, the expression levels of CD80(C), CD86 (FIG. 7D) and CD80 CD86 double positive (FIG. 7E). (Flow cytometric analysis of surface marker expression following 24-hour co-culture with CD19 CAR-T cells at different effector-to-target ratios. Quantification of CD80 (FIG. 7C), CD86 (FIG. 7D), and CD80/CD86 double-positive populations (FIG. 7E) in residual B cells). FIG. 7C-E were analyzed using an unpaired t-test. P value<0.05=*, <0.01=**, <0.001=***, <0.0001=****, P>0.05=no significance=NS.

FIGS. 8A-8D demonstrates the expression specificity of prostatic acid phosphatase (PAP) in normal and tumor tissues. Analysis of the GTEx dataset reveals that PAP expression is highly restricted to prostate tissue in healthy individuals. TCGA data show elevated PAP expression in prostate tumor samples compared to other cancer types, with a clear differential between prostate adenocarcinoma (PRAD) tumors and matched normal tissues. Single-cell RNA sequencing of human prostate tumors confirms that PAP expression is localized primarily to the epithelial compartment. Immunohistochemistry further validates strong PAP protein expression in prostate tumor tissues, with minimal or no detectable expression in non-prostate control tissues. Comparative IHC scoring highlights the superior tumor specificity of PAP relative to other targets such as PSMA and CD46.

The subject matter described above is provided by way of illustration only and should not be construed as limiting. Various modifications and changes may be made to the subject matter described herein without following the example embodiments and applications illustrated and described, and without departing from the true spirit and scope of the present disclosure, which is set forth in the following claims.

All publications, patents and patent applications cited in this specification are incorporated herein by reference in their entireties as if each individual publication, patent, or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method of expanding a solid tumor-targeting chimeric antigen receptor T (CAR-T) cell population in a subject, the method comprising:
co-administering to the subject:
a) a first CAR-T cell population comprising T cells expressing a chimeric antigen receptor that specifically binds a solid tumor antigen;
b) a second CAR-T cell population comprising T cells expressing a chimeric antigen receptor that specifically binds CD19; and
c) B cells expressing CD19;
wherein the expansion of the first CAR-T cell population occurs in vivo in the absence of exposure to its cognate tumor antigen and is mediated by costimulatory signals and cytokine signals derived from the interaction of the second CAR-T cell population with the B cells.

2. The method of claim 1, wherein the solid tumor antigen is transmembrane prostatic acid phosphatase (tmPAP).

3. The method of claim 1, wherein the chimeric antigen receptor of the first CAR-T cell population comprises a single-chain variable fragment (scFv) derived from a human antibody that binds specifically to tmPAP.

4. The method of claim 1, wherein the chimeric antigen receptor of the second CAR-T cell population comprises a 4-1BB costimulatory domain and a CD3ζ signaling domain.

5. The method of claim 1, wherein the B cells are administered to the subject at multiple time points following the administration of the first and second CAR-T cell populations.

6. The method of claim 1, wherein the costimulatory signals comprise CD80 and CD86 expressed on the surface of B cells.

7. The method of claim 1, wherein the cytokine signals comprise at least one of IL-2, IL-15, IL-3, IL-4, or CXCL9 secreted by the B cells or the second CAR-T cell population.

8. The method of claim 1, wherein the expansion of the first CAR-T cell population results in a T cell phenotype characterized by increased expression of CCR7, IL7R, and TCF7, and decreased expression of PD-1 and LAG-3.

9. The method of claim 1, wherein the first CAR-T cell population retains a central memory (TCM) or stem-cell memory (T_SCM) phenotype after expansion.

10. The method of claim 1, wherein the B cells are obtained from the subject's peripheral blood and isolated using a B cell selection kit.

11. The method of claim 1, wherein the subject is a human diagnosed with metastatic or treatment-refractory prostate cancer.

12. The method of claim 1, wherein the expansion of the first CAR-T cell population is not accompanied by activation of T cell receptor (TCR) or CD3ζ signaling in the first CAR-T cell population.

13. The method of claim 1, wherein the second CAR-T cell population and the B cells are administered at an effector-to-target ratio of between 1:1 and 1:10.

14. The method of claim 1, wherein the expansion of the first CAR-T cell population is associated with increased antitumor efficacy in a solid tumor xenograft model.

15. The method of claim 1, wherein the method further comprises measuring cytokine levels in the peripheral blood of the subject after treatment to monitor CAR-T cell expansion.

16. A method for reducing treatment-associated adverse events in a subject receiving chimeric antigen receptor T (CAR-T) cell therapy, the method comprising:
administering to the subject a first population of CAR-T cells that target a solid tumor antigen;
administering to the subject a second population of CAR-T cells that target CD19; and
co-administering B cells expressing CD19;
wherein the expansion of the first population of CAR-T cells occurs in vivo without stimulation of T cell receptor (TCR) or CD3ζ signaling in the first population, and
wherein the first population of CAR-T cells exhibits a memory-like phenotype and reduced expression of exhaustion markers, thereby reducing the incidence or severity of cytokine release syndrome or immune effector cell-associated neurotoxicity in the subject.

17. The method of claim 16, wherein the first population of CAR-T cells expresses a chimeric antigen receptor comprising a fully human scFv that binds transmembrane prostatic acid phosphatase (tmPAP).

18. A system for expanding a solid tumor-targeting chimeric antigen receptor T (CAR-T) cell population in a subject, the system comprising:
a) a first CAR-T cell population comprising T cells engineered to express a chimeric antigen receptor that specifically binds a solid tumor antigen;
b) a second CAR-T cell population comprising T cells engineered to express a chimeric antigen receptor that specifically binds a surface antigen expressed on circulating lymphocytes; and
c) an auxiliary cell population comprising lymphocytes expressing the surface antigen bound by the second CAR-T cell population;
wherein the expansion of the first CAR-T cell population in vivo is driven by costimulatory signals and cytokine signals resulting from the interaction of the second CAR-T cell population with the auxiliary cell population, and
wherein the expansion occurs in the absence of cognate antigen engagement by the first CAR-T cell population.

19. The system of claim 18, wherein the surface antigen is CD19, and the auxiliary cell population comprises autologous B cells expressing CD19.

20. The system of claim 18, wherein the first CAR-T cell population comprises a CAR with a fully human single-chain variable fragment (scFv) that binds transmembrane prostatic acid phosphatase (tmPAP).
